(19) United States
(12) Patent Application Publication
(10) Pub. No.: US 2009/0306045 A1
(43) Pub. Date: Dec. 10, 2009

(54) Inhibition of Glycogen Synthase Kinase and Methods of Treating Autoimmune or Immune Inflammatory Disease

(76) Inventors: Ira Mellman, San Francisco, CA (US); Aimin Jiang, New Haven, CT (US)

Correspondence Address: COLEMAN SUDOL SAPONE, P.C., 714 COLORADO AVENUE, BRIDGE PORT, CT, 06605-1601, US

(21) Appl. No.: 11/991,654

(22) PCT Filed: Dec. 22, 2006
(86) PCT No.: PCT/US2006/048827

§ 371 Date: Mar. 28, 2008

Related U.S. Application Data
(60) Provisional application No. 60/753,034, filed on Dec. 22, 2005.

(51) Int. Cl. A61K 31/55; A61K 31/4015; A61P 37/00

(52) U.S. Cl. 514/212.06; 514/422

(57) ABSTRACT

The present invention relates to the use of glycogen synthase kinase 3(GSK3) inhibitors, especially inhibitors of GSK-3α, GSK-3β and GSK-3β2, preferably, inhibitors of GSK-3β, in patients having autoimmune diseases and/or immune dysfunction/dysregulation to induce immune tolerance. Inhibition of GSK leads to activation of a pathway of dendritic cell maturation which leads to a dendritic phenotype which attenuates, rather than induces, immune responses. The immune responses and mature dendritic cells produced by the method of the present invention redirect or attenuate the immune response in individuals, thus leading to effective therapies for a number of autoimmune diseases and/or diseases of immune dysfunction/dysregulation (immune inflammatory diseases), including systemic lupus erythematosus (SLE), autoimmune diabetes (type I diabetes mellitus), asthma, rheumatoid arthritis, inflammatory bowel disease, among numerous others.

### RELATED APPLICATIONS

This application claims the benefit of priority of U.S. provisional application US60/753,034, filed Dec. 22, 2005, the entire contents of which are incorporated by reference herein.

This invention was made with support from the United States government under grant no. NIH R37-A134098 and from the Ludwig Institute for Cancer Research Consequently, the government retains certain rights in the invention.

### FIELD OF THE INVENTION

The present invention relates to the use of glycogen synthase kinase 3(GSK3) inhibitors, especially inhibitors of GSK-3α, GSK-3β and GSK-3β2, preferably, inhibitors of GSK-3β, in, for example, dendritic cells in the immune system. Inhibition of GSK leads to activation of a pathway of dendritic cell maturation which leads to a dendritic phenotype which attenuates, rather than induces, immune responses. The immune responses and mature dendritic cells produced by the method of the present invention redirect or attenuate the immune response in individuals, thus leading to effective therapies for a number of autoimmune diseases and/or diseases of immune dysfunction/dysregulation (immune inflammatory diseases), including systemic lupus erythematosus (SLE), autoimmune diabetes (type I diabetes mellitus), asthma, rheumatoid arthritis, inflammatory bowel disease, among numerous others.

### BACKGROUND OF THE INVENTION

Dendritic cells (DCs) reside at the interface of innate and adaptive immunity. As the sentinels of the immune system, immature DCs are distributed in peripheral tissues where they continuously sample the environment by endocytosis (Banchereau and Steinman, 1998). Upon encountering pathogens or a variety of pro-inflammatory mediators, DCs commence a complex and heterogeneous transformation process termed “maturation”, which greatly enhances their capacity for antigen processing and presentation. Maturation may occur prior to, during or after migration to secondary lymphoid organs where the DCs serve to prime naïve T cells (Banchereau and Steinman, 1998). The general features of DC maturation are well understood (Mellman and Steinman, 2001) and involve the translocation of MHC class II molecules (MHCII) from lysosomal compartments to the plasma membrane, the upregulation of costimulatory molecules such as CD80 and CD86, the activation of lysosomal antigen processing, and the release of a host of immunostimulatory cytokines (Trombetta and Mellman, 2005). There is also a marked increase in the expression of lymphoid chemokine receptors such as CCR7, required for directed migration of DCs to lymph nodes (Randolph et al., 2005). Maturation is most often thought of as being triggered by activation of one or more Toll-like receptors (TLRs), although a variety of pro-inflammatory mediators and T cell products can also induce DCs to mature (Mellman and Steinman, 2001; Trombetta and Mellman, 2005).

Although the phenotypic correlates of DC maturation are clear, their relationship to DC function is complex. For example, depending on the type of microbial stimulus, DCs can prime qualitatively different types of effector T cell responses (Lanzavecchia and Sallusto, 2001). In addition, DCs play a role in maintaining tolerance to self proteins (Steinman et al., 2003). Precisely how DCs accomplish this latter task is unclear, but is thought to involve ingestion of apoptotic cells in peripheral tissues and the presentation of captured self antigens in lymph nodes in a fashion that results in transient stimulation and death of autoreactive T cells (Steinman et al., 2003; Steinman et al., 2000). The maturation state, origin, and phenotype of these “tolerogenic DCs” remain poorly understood.

Recent work has suggested that the features associated with DC maturation can be quite variable. For example, DC maturation and migration to lymph nodes can be independently regulated (Geissmann et al., 2002; Verbovetski et al., 2002), although the underlying mechanisms have not been elucidated. In DCs lacking the TLR adaptor MyD88, the phenotypic maturation of DCs can occur without inflammatory cytokine production (Kaisho et al., 2001). Such DCs cannot activate naïve CD4 T cells in vivo suggesting that this phenotype, should it occur physiologically, might play a role in tolerance (Pasare and Medzhitov, 2004). Indeed, DCs matured by inflammatory cytokines in the absence of TLR agonists may not be able to fully prime CD4 T cell immunity (Lutz and Schuler, 2002; Sporri and Reis e Sousa, 2005).

Can DCs initiate maturation in the absence of inflammatory or microbial stimuli? DCs of the skin, particularly epidermal Langerhans cells (LCs), present an intriguing example. LCs form networks anchored to neighboring keratinocytes via E-cadherin, a component of epithelial cell junctions that is also expressed by LCs (Jakob et al., 1999; Tang et al., 1993). Although these networks are quite stable, LCs appear to traffic to lymph nodes, with their rate of emigration being enhanced by TV exposure or mechanical trauma (Jakob et al., 2001; Merad et al., 2002). How this occurs is unknown, but seems likely to require the disruption of E-cadherin interactions. In epithelial cells, E-cadherin forms a complex with members of the catenin family, which control interactions with the actin cytoskeleton and (after translocation to the nucleus) act as cofactors for TCF/LEF transcriptional activators (Vasioukhin and Fuchs, 2001). Given these functions, the amount of free cytosolic catenins, especially β-catenin, is carefully regulated. Under resting conditions, the bulk of β-catenin is sequestered to the E-cadherin cytoplasmic domain, with the cytosolic pool further attenuated by its phosphorylation by glycogen synthase kinase 3β (GSK3β and subsequent proteosomal degradation (Nelson and Nusse, 2004; Staal and Clevers, 2005). Activation of Wnt signaling activates TCF/LEF-dependent transcription by increasing free β-catenin due in part to an inhibition of GSK3β.

In LCs, it is unclear whether E-cadherin-mediated cell-cell adhesion is linked to activation of β-catenin signaling, although early work demonstrated that disruption of LC-LC interactions in vitro could trigger phenotypic maturation (Jakob and Udey, 1998; Riedl et al., 2000a; Riedl et al., 2000b). We find that the E-cadherin/β-catenin expression is not limited to LCs, and that activation of this pathway can trigger a functionally distinct pathway of maturation that appears more closely linked to maintaining tolerance than to initiating immunity.

The maturation of dendritic cells (DCs) following exposure to microbial products or inflammatory mediators plays a critical role in initiating the immune response. We now find that maturation can also occur under steady state conditions, triggered by alterations in E-cadherin-mediated DC-DC adhesion. Selective disruption of these interactions induces the typical features of DC maturation including the upregulation of costimulatory molecules, MHC class II, and chemokine receptors. These events were triggered at least in part by activation of the β-catenin pathway. However, unlike maturation induced by microbial stimulation of Toll-like receptors, E-cadherin-stimulated DCs failed to release immunostimulatory cytokines. As a result, E-cadherin-stimulated DCs elicited an entirely different T cell response in vivo, generating T cells with a regulatory as opposed to an effector phenotype. Thus, DC matured by alteration in E-cadherin-mediated adhesion may contribute to the elusive population of “tolerogenic DCs” produced in vivo under steady state conditions, which help prevent immune responses to self antigens.

### BRIEF DESCRIPTION OF THE FIGURES

FIG. 1 shows numerous GSK3 inhibitor compounds which are useful in the present invention.

FIG. 2. Disruption of E-Cadherin-Mediated Clusters Results in DC Maturation

(2A) DCs matured after cluster disruption (CD) exhibited similar morphological changes as induced by LPS. DCs matured by CD or LPS were labeled for MHC II (first column) and the lysosomal marker Lamp 2 (second column).

(2B) Anti-E-cadherin antibodies can block DC maturation induced by CD. BMDCs were prepared as described and CD11c⁺ DCs were purified at day 6 and replated at 5×10⁵ cells/ml. Treatment of an anti-E-cadherin mAb (Sigma) but not isotype-matched anti-CD11b mAb or mouse IgG inhibited the upregulation of CD86.

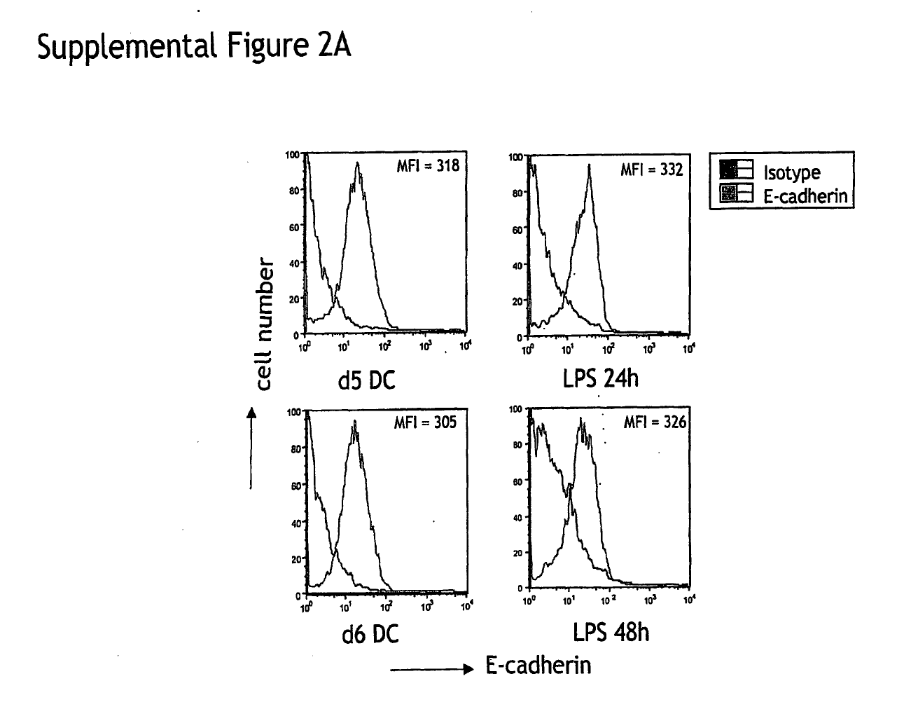

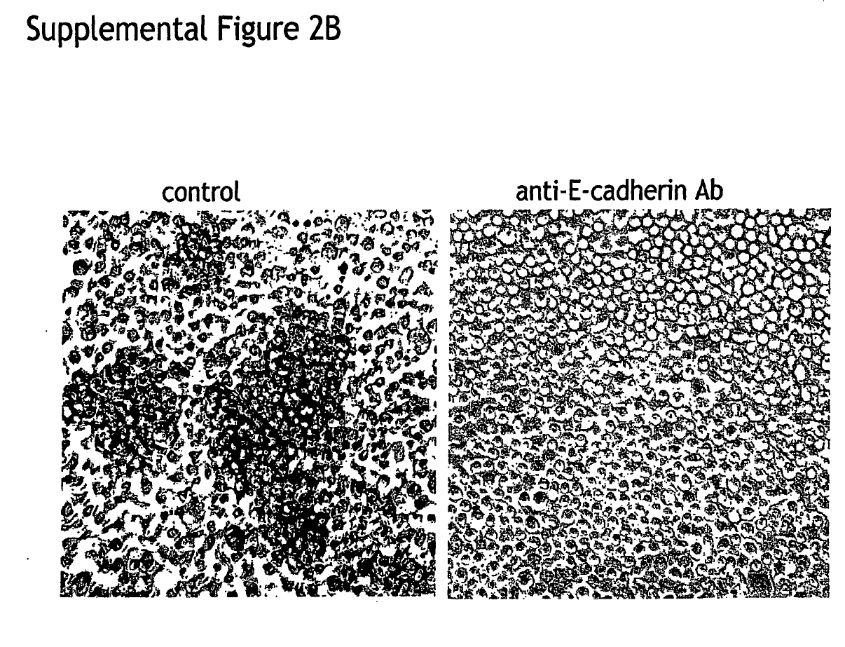

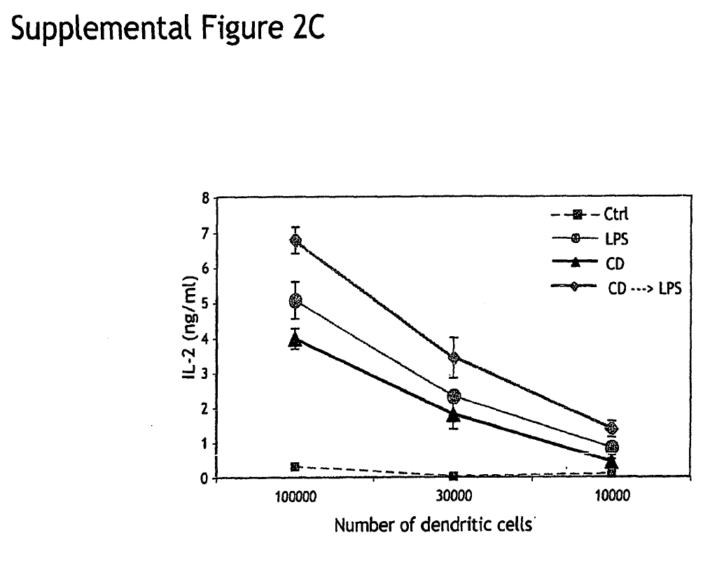

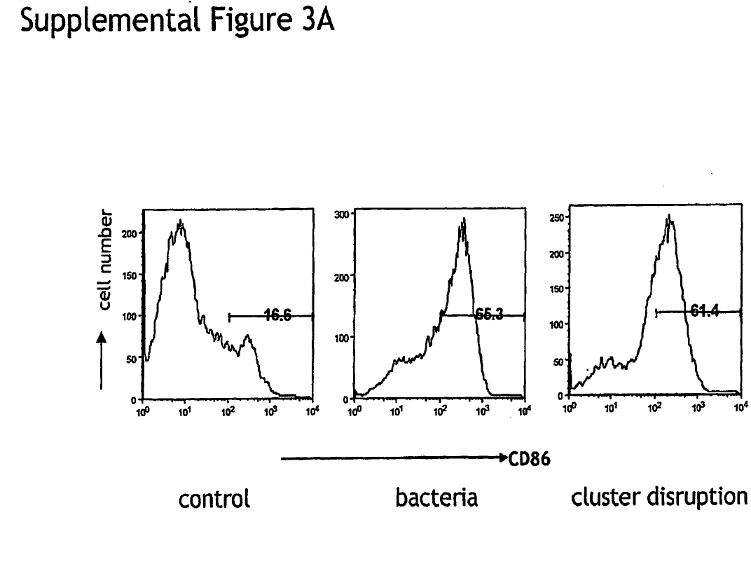

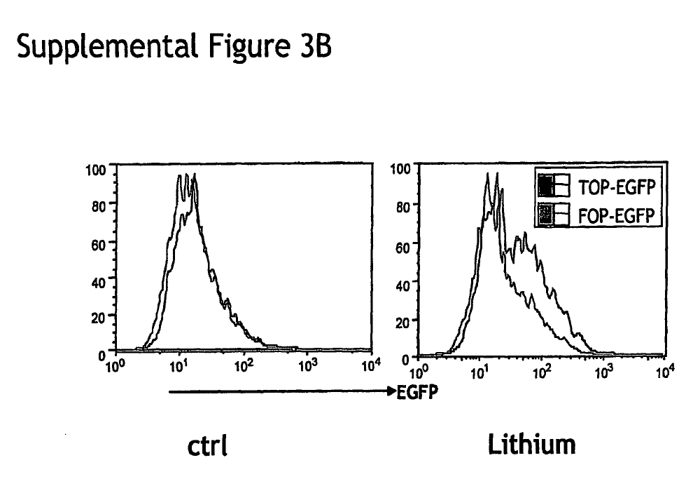

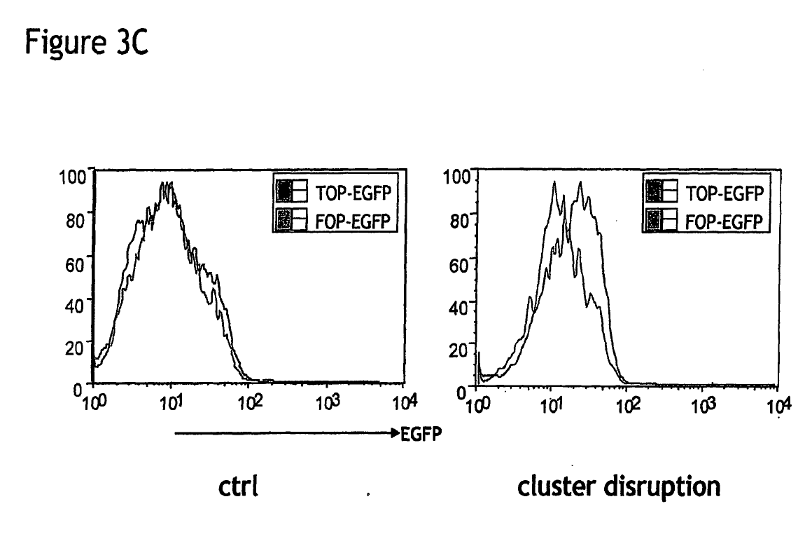

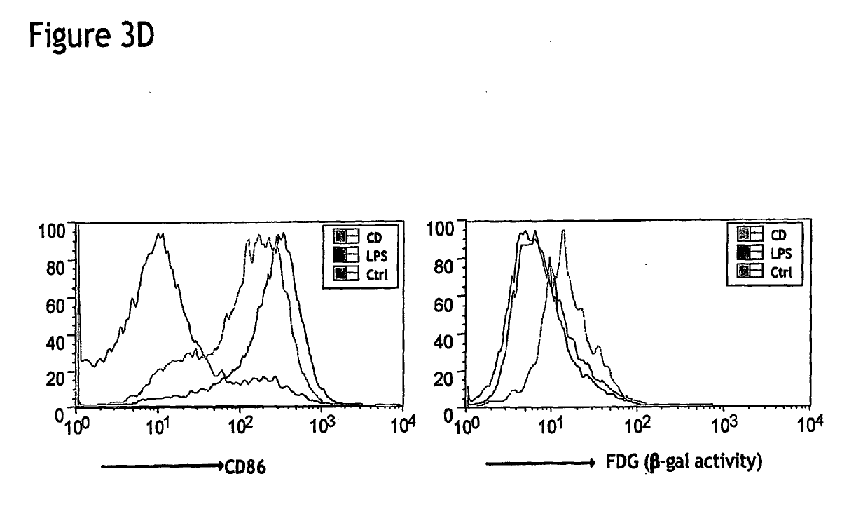

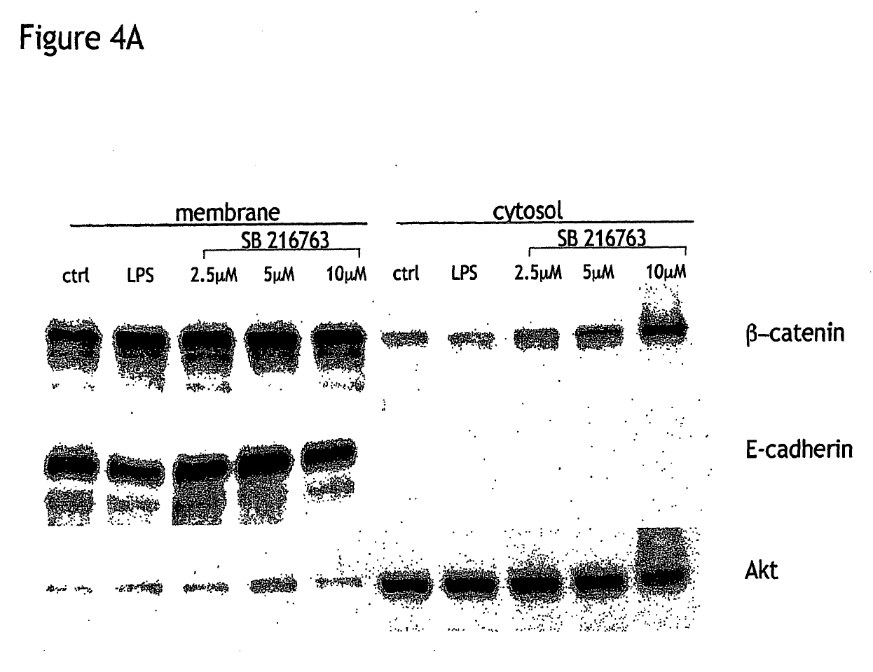

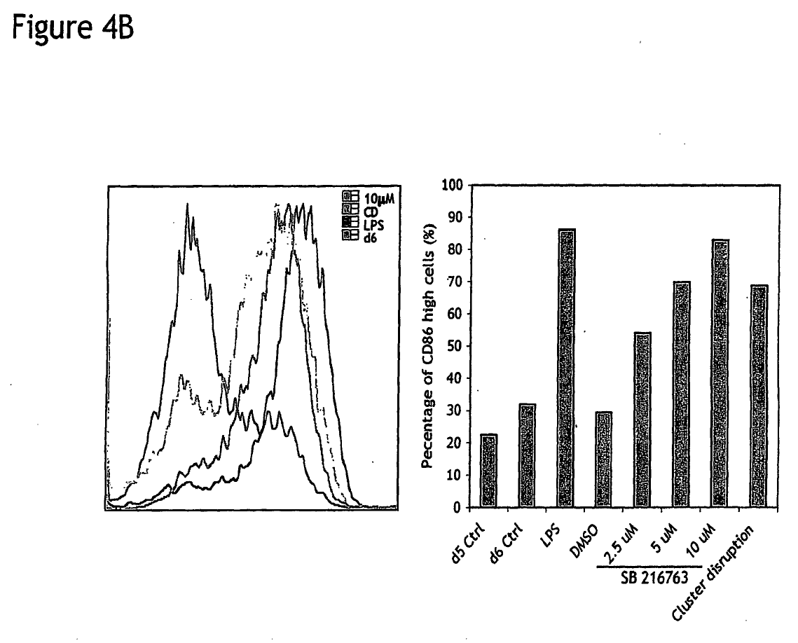

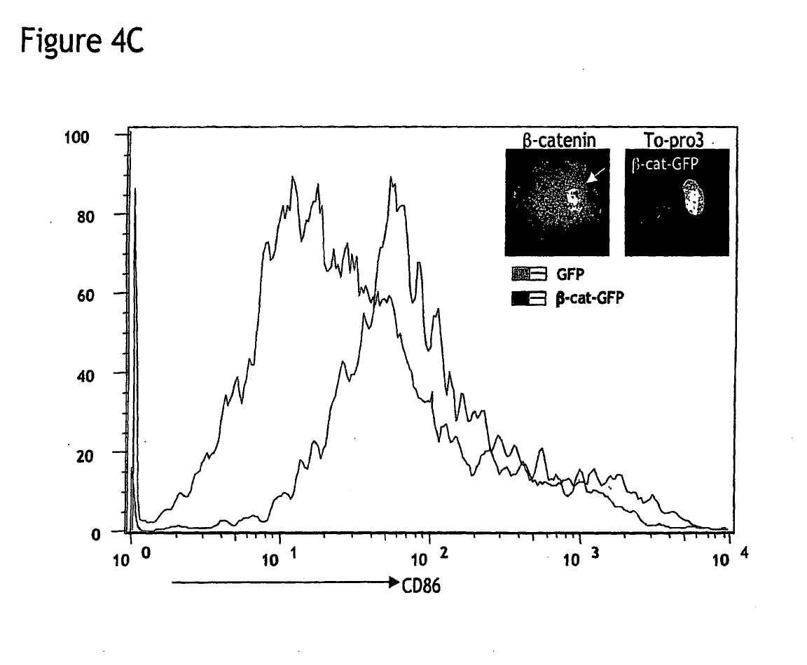

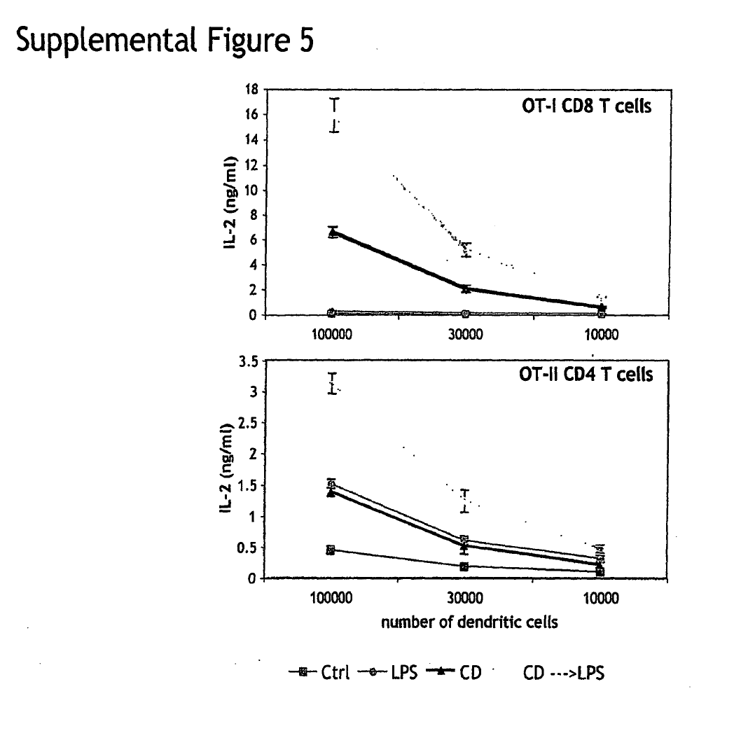

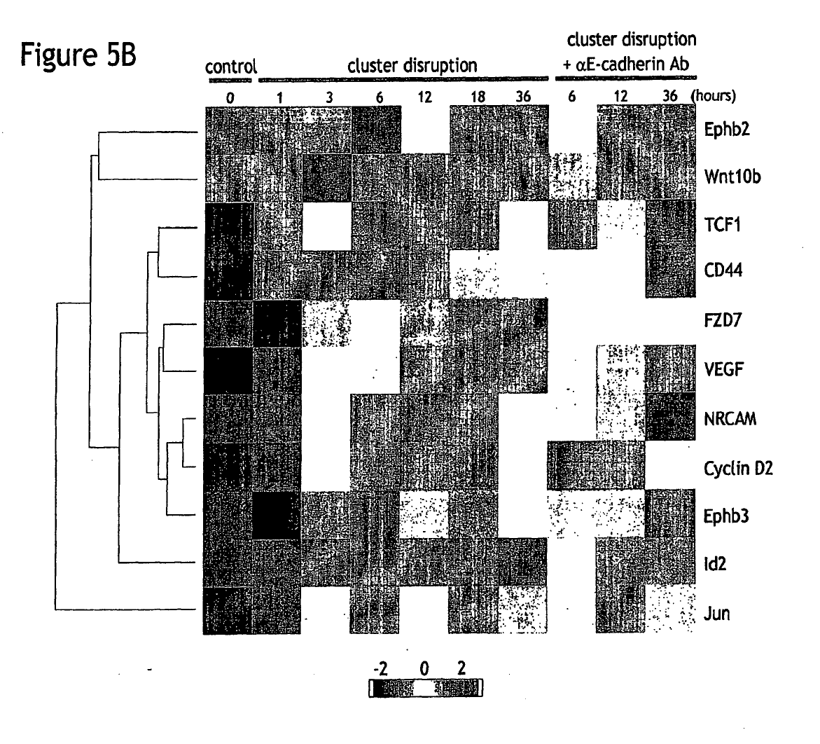

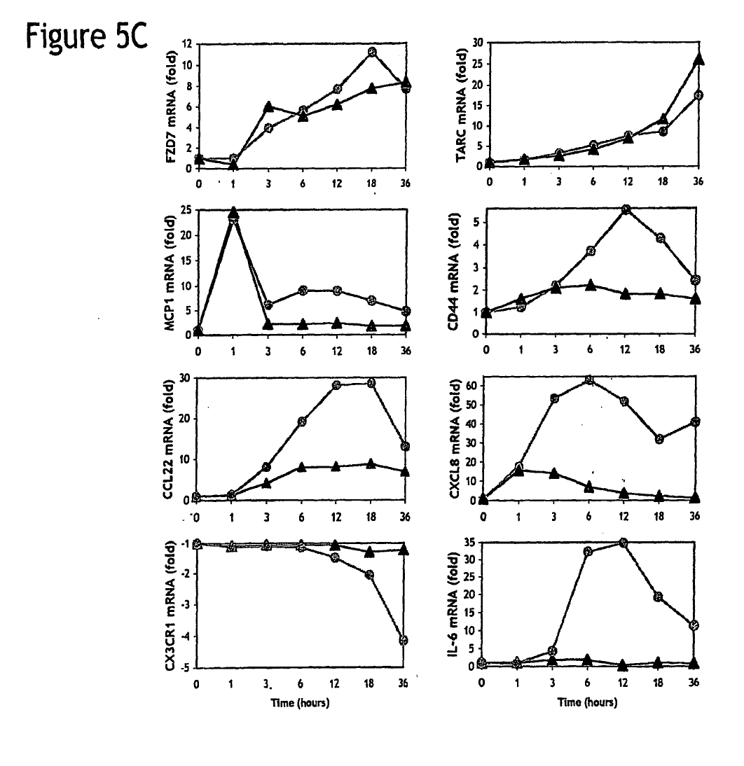

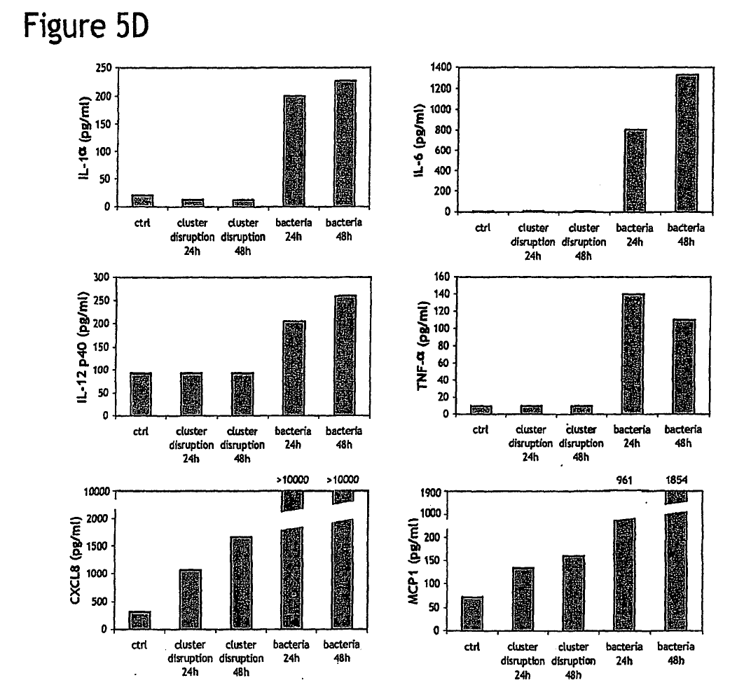

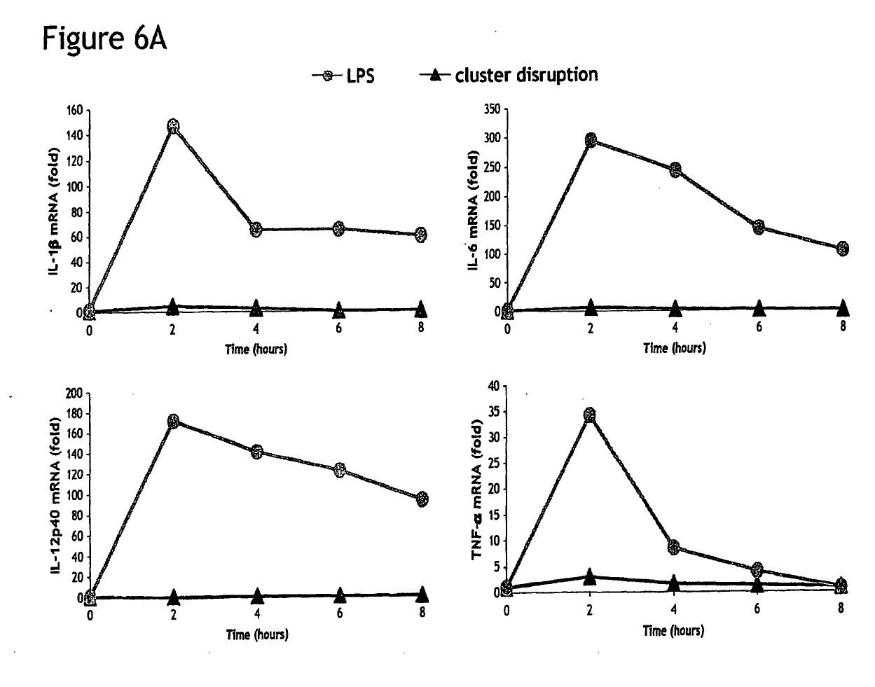

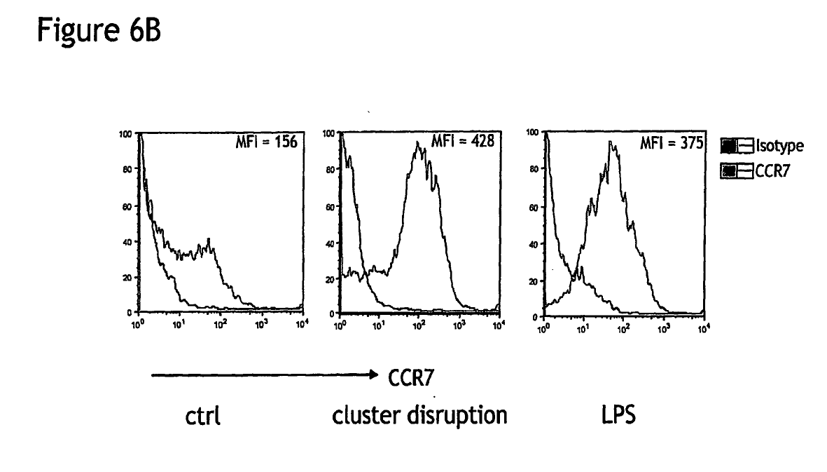

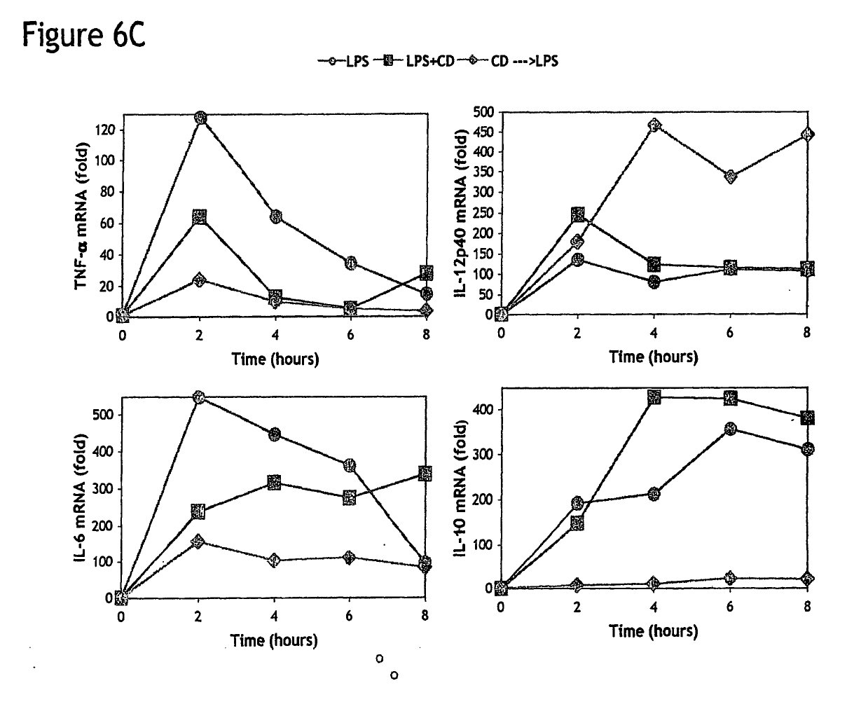

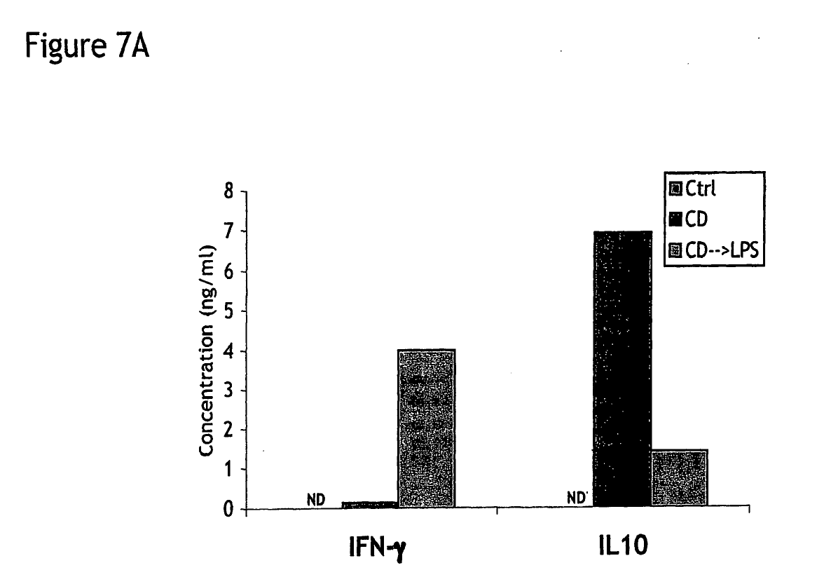

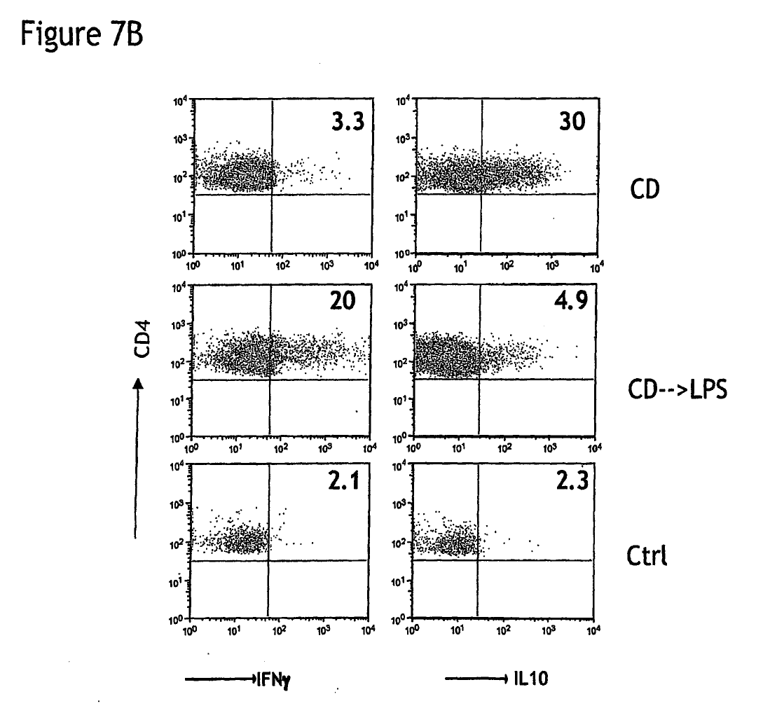

Supplemental FIG. 2. E-Cadherin Mediated DC-DC Contact in Mouse BMDC and Cluster Disruption (CD) Led to Mature DCs Capable of Antigen Presentation

(Supp. 2A) E-cadherin was expressed by murine BMDCs. Gated CD11c⁺ cells were analyzed for their surface expression of E-cadherin. Mean fluorescence intensity (MFI) was shown for surface E-cadherin staining.

(Supp. 2B). Addition of anti-E-cadherin antibodies inhibits cluster formation of BMDCs. DCs cultured in 96 well plate were either untreated or treated with an E-cadherin Ab, and cluster formation was checked 24 hours later.

(Supp. 2C). Activation of Naïve CD4 T cells by CD-matured DCs. OVA peptide (323-339) pulsed untreated, LPS-stimulated or cluster disruption (CD)-matured CD11c⁺ DCs were mixed with naïve CD4 T cells from OT-II lymph nodes and incubated for ˜28 hours.

FIG. 2b Complete. CD as Well as Treatment with the GSK3β Inhibitor Resulted in Activation of β-Catenin

Bone marrow-derived DC cultures were either treated with LPS, SB216763 or cluster disruption (CD). CD11c⁺ cells were purified and same number of cells were used to make cell lysates as described. Cell lysates were then subject to sequential immunoprecipitation with antibodies against E-cadherin and β-catenin, followed by immunoblotting with antibodies against E-cadherin (top), active β-catenin (middle) and total β-catenin (bottom).

FIG. 3. Disruption of the E-Cadherin-Mediated Adhesion Activates a Distinct β-Catenin/TCF Signaling Pathway Independent of TLR Signaling

(3A) CD did not activate NF-κB and p38 MAPK signaling pathways. Cell lysates from different treatments were analyzed by immunoblotting with anti-phospho-p38 MAPK Ab (top), phosphorylation-specific Ab against IκBα (middle) and anti-tubulin Ab (bottom).

(3B) CD resulted in activation of β-catenin. BMDCs were either treated with LPS or CD and cell lysates from CD11c⁺ DCs were subject to sequential immunoprecipitation with antibodies against E-cadherin and β-catenin, followed by immunoblotting with antibodies against E-cadherin (top), active β-catenin (middle) and total β-catenin (bottom).

(3C) CD resulted in β-catenin/TCF mediated transcription. BMDC cultures were transfected with pLTRH1 containing the TOP-EGFP or FOP-EGFP at day 2 and transfected cells were purified with magnetic columns at day 6. EGFP was measured on CD 11c⁺ DCs immediately after purification (control) or 48 hr later (CD) by FACS.

(3D) CD but not LPS treatment led to transactivation of TOPgal reporter. BMDCs from transgenic TOPGAL reporter mice were matured by LPS or CD, β-galactosidase activity was measured by flow cytometry using fluorescein di-β-D-galactosidase (FDG) as a substrate.

Supplemental FIG. 3. CD Led to Maturation of TLR4^{-/-} DCs and Activation of □̅-Catenin Signaling Pathway by Lithium Resulted in TCF-Dependent EGFP Expression in MDCK Cells

(Supp. 3A) CD led to phenotypical maturation of TLR4^{-/-} DCs. TLR4^{-/-} DCs are either treated with bacteria or cluster disruption for 24 hours and then subject to FACS analysis for CD86 expression.

(Supp. 3B) MDCK cells transfected with pLTRH1 containing the TOP-EGFP or FOP-EGFP were either untreated or treated with LiCl (20 mM) for 2 days. EGFP was measured on CD4⁺ transfected cells before or after the treatment by FACS.

FIG. 4. Activation of β-Catenin Signaling Pathway Induces DC Maturation

(4A) Dose-dependent accumulation of cytosolic β-catenin after treatment with GSK3β inhibitor SB216763. BMDCs were treated with either LPS or different doses of SB216763. CD11c⁺ DCs were then fractionated into membrane and cytosolic fractions, followed by immunoblotting with antibodies against β-catenin (top) and E-cadherin (middle). Akt was probed as a loading control (bottom).

(4B) Inhibition of GSK3β results in DC maturation. CD11c⁺ DCs after different stimuli were subject to FACS analysis. The left histogram overlay shows a representative FACS profile of CD86 expression for each condition, with SB216763 at 10 μm CD86^{high} cells represent mature DCs on the right.

(4C) Expression of β-catenin enhanced spontaneous DC maturation. BMDC cultures were transfected either with GFP or β-catenin-GFP and were subject to FACS analysis for CD86 expression at day 6. Expression of β-catenin-GFP but not GFP induced CD86 upregulation, although not as strongly as after CD or drug treatment. Insert: β-catenin translocates to the nucleus. 12 hr after CD of DCs expressing β-catenin-GFP, cells were fixed, labeled with a β-catenin antibody and the DNA dye TO-Pro3, and imaged by confocal microscopy. β-catenin was clearly translocated into the nucleus (arrow).

FIG. 5. CD-Matured Human DCs Failed to Produce Inflammatory Cytokines

(5A) More than 700 genes were differentially regulated upon maturation by either CD or bacterial stimulation. Heatmap was generated as detailed in the Experimental Procedures.

(5B) CD led to upregulation of 10 direct β-catenin/TCF target genes. Target genes were selected according to R. Nusse and colleagues and heatmap was created as described in Experimental Procedures. Wnt10b was not a target gene but was included for comparison.

(5C) Representative gene expression profiles were plotted from the microarray data.

(5D) Human CD34⁺ DCs matured by CD did not produce inflammatory cytokines. Luminex assays for multiple cytokines and chemokines were performed on supernatants from CD or bacteria-matured DCs. One of two independent experiments is shown.

FIG. 6. CD-Matured Murine BMDCs Upregulated CCR7 without Inflammatory Cytokine Induction

(6A) CD-matured murine BMDCs did not induce inflammatory cytokines IL-1β, IL-6, IL-12p40 and TNFα. Real-time RT-PCRs were performed on total RNA isolated from DCs treated with either LPS or CD for the indicated times, the expression of each gene then was normalized to β-actin expression.

(6B) CD-matured BMDCs express elevated level of surface CCR7. DCs untreated or matured by either CD or LPS were subjected to FACS analysis.

(6C) Addition of LPS after cluster disruption synergistically enhances or inhibits cytokine production. Real-time RT-PCRs were performed and analyzed as described in Panel A. Cluster-disrupted DCs were stimulated with LPS simultaneously (CD+LPS) or LPS was added 14-18 hr afterwards for the indicated times (CD--->LPS). Results from one of three different sets of samples are shown.

FIG. 7. DCs Matured Sequentially by CD and LPS Primed Naïve CD4 Cells to Become IFN-γ-Producing Effectors but DCs Matured by CD Alone Instead Generated IL10-Producing CD4 T Cells

(7A) Immunization with DCs matured by CD alone induced T cells that produced IL10 instead of IFN-γ. CD11c⁺ BMDCs were purified at day 6-7 of culture and pulsed with OVA peptide 323-339 (10 μg/ml) for 2 hr and washed extensively before resuspension in PBS. 1-2.5×10⁶ DCs were injected intravenously into C57BL/6 mice at day 0, 2 and 4. Splenocytes (1×10⁶ cells/well) were prepared at day 7 and stimulated with antigens for 3 days. The supernatants were collected and cytokines were measured with the Luminex assays.

(7B) DC matured by CD generated IL10-producing CD4 T cells instead of IFN-γ-producing effector cells. BFA (5 μg/ml) were added for 6 hr at the end of 2-3 day restimulation and splenocytes were then stained as described. The numbers indicate the percentage of IFN-γ or IL10-positive cells of gated CD4⁺CD25⁺ cells. Results are representative of four similar experiments each consisted of two mice for CD and CD->LPS treatments.

### OBJECTS OF THE INVENTION

It is an object of the invention to provide methods for inhibiting glycogen synthase kinase 3 (“GSK3”), including one or more of its isoforms: GSK-3α, GSK-3β and GSK-3β2 in dendritic cells of a patient or subject.

It is another object of the invention to inhibit GSK3, especially one or more of GSK-3α, GSK-3β and GSK-3β2 in dendritic cells of a patient or subject to activate an E-cadherin/β-catenin pathway in dendritic cells to produce mature dendritic cells which exhibit T cell response associated with induction or maintenance of T cell “tolerance”, rather than immunity.

It is still another object of the invention to provide a method of treating autoimmune disease in a patient or subject by administering to the patient or subject in need of therapy an effective amount of a GSK3 inhibitor, including an inhibitor of GSK-3α, GSK-3β and GSK-3β2 alone or in combination with another agent to treat autoimmune disease.

### BRIEF DESCRIPTION OF THE INVENTION

The present invention relates to the discovery that the inhibition of glycogen synthase kinase 3 enzyme (GSK3), especially one or more of GSK-3α, GSK-3β and GSK-3β2 in dendritic cells of a patient or subject, activates the E-cadherin/β-catenin pathway in those dendritic cells to produce mature dendritic cells which exhibit T cell response associated with induction or maintenance of T cell “tolerance” (“immune tolerance”), rather than immunity. Thus, the administration of an inhibitor of GSK3, preferably an inhibitor of GSK-3α, GSK-3β or GSK-3β2, most preferably an inhibitor of GSK-3β in an effective amount of a patient or subject, results in the activation of the E-cadherin/β-catenin pathway in those dendritic cells and the production of mature dendritic cells which exhibit a T cell response associated with the induction or maintenance of T cell tolerance in said patient.

In another aspect of the invention, a method of treating autoimmune disease in a patient comprises administering at least one GSK3 inhibitor to a patient in need of therapy for an autoimmune disease comprising administering an effective amount of a GSK3 inhibitor, preferably an inhibitor of GSK-3α, GSK-3β and/or GSK-3β2, preferably an inhibitor of GSK-3β to said patient to treat the autoimmune disease. In aspects of the present invention, autoimmune diseases include systemic lupus erythematosus (SLE), diabetes mellitus (type I), asthma, Grave's disease, arthritis, including rheumatoid arthritis and osteoarthritis, pernicious anemia, and multiple sclerosis, among numerous others. In other aspects of the invention, an autoimmune disease other than diabetes type I is treated using a GSK3 inhibitor, preferably a GSK3β inhibitor, as otherwise described herein. Numerous autoimmune diseases may be treated using the method of the present invention including autoimmune blood diseases, including pernicious anemia, autoimmune hemolytic anemia, aplastic anemia, idiopathic thrombocytopenic purpura, ankylosing spondylitis; autoimmune diseases of the musculature including polymyositis and dermatomyositis, autoimmune diseases of the ear including autoimmune hearing loss and Meniere's syndrome, autoimmune eye diseases, including Mooren's disease, Reiter's syndrome and Vogt-Koyanagi-Harada disease, autoimmune diseases of the kidney including glomerulonephritis and IgA nephropathy, diabetes mellitus (type I); autoimmune skin diseases including pemphigus (autoimmune bullous diseases), such as pemphigus vulgaris, pemphigus foliaceus, pemphigus erythematosus, bullous pemphigoid, vitiligo, epidermolysis bullosa acquisita, and alopecia areata, cardiovascular autoimmune diseases, including autoimmune myocarditis, vasculitis including Churg-Strauss syndrome, giant cells arteritis, Kawasaki's disease, polyarteritis nodosa, Takayasu's arteritis and Wegener's granulomatosis; endocrine autoimmune diseases, including Addison's disease, autoimmune hypoparathyroidism, autoimmune hypophysitis, autoimmune oophoritis, autoimmune orchitis, Grave's Disease, Hashimoto's thyroiditis, polyglandular autoimmune syndrome type 1 (PAS-1) polyglandular autoimmune syndrome type 2 (PAS-2), and polyglandular autoimmune syndrome type 3 (PAS-3); autoimmune gastroenteric diseases including autoimmune hepatitis, primary biliary cirrhosis, inflammatory bowel disease, celiac disease, Crohn's disease; autoimmune nervous diseases, including multiple sclerosis, myasthenia gravis, Guillan-Barre syndrome and chronic inflammatory demyelinating neuropathy; and systemic autoimmune diseases including systemic lupus erythematosus, antiphospholid syndrome, autoimmune lymphoproliferative disease, autoimmune polyendocrinopathy, Bechet's disease, Goodpasture's disease, arthritis, including rheumatoid arthritis, osteoarthritis and septic arthritis, sarcoidosis, scleroderma and Sjogren's syndrome.

In treating autoimmune diseases according to the present invention, at least one GSK3 inhibitor in an effective amount, optionally in combination with a pharmaceutically acceptable carrier, additive or excipient is administered to a patient in need of such treatment to provide a favorable disposition of the disease state. In preferred embodiments, the GSK3 inhibitor is an inhibitor of GSK3, preferably an inhibitor of one or more of GSK-3α, GSK-3β and/or GSK-3β2, preferably an inhibitor of GSK-3β. Efficacious therapies may also require the simultaneous administration of the antigen or antigens that are the causative or sustaining targets of the autoimmune or chronic inflammation.

### DETAILED DESCRIPTION OF THE INVENTION

The following terms are used throughout the present specification to describe the invention.

The term “patient” or “subject” refers to an animal, preferably a mammal, even more preferably a human, in need of treatment or therapy to which GSK3 inhibitors according to the present invention are administered in order to treat an autoimmune disease, especially a condition or disease state associated with an autoimmune disease as otherwise described herein.

The term “compound” is used herein to refer to any specific chemical compound disclosed herein. Within its use in context, the term generally refers to a single compound, generally a small molecule inhibitor of GSK3.

The term “glycogen synthase kinase 3” is used to describe a serine/threonine protein kinase. Glycogen synthase kinase-3 (GSK-3) is a serine/threonine protein kinase encoded by two highly homologous and ubiquitously expressed genes. The catalytic domains of mammalian GSK-3K and GSK-3L are 95% identical at the amino acid level, whereas the amino- and carboxy-termini are less conserved See Woodgett, EMBO J. 9, 2431-2438 (1990).

GSK-3 was originally identified by virtue of its ability to phosphorylate and inactivate glycogen synthase, the rate limiting enzyme in glycogen synthesis. However, it is now apparent that GSK-3 has many putative targets, including IRS-1, the translation initiation factor eIF2B, transcription factors c-jun, CREB, NFAT, β-catenin, C/EBPK and the neuronal microtubule associated proteins MAP-1B and Tau.

A variety of extracellular stimuli indirectly inhibit cellular GSK-3 activity, including insulin, growth factors, Wnt cell specific proteins and cell adhesion. Since these stimuli elicit a diverse range of responses in a number of different cell types, inhibition of GSK-3 activity is potentially pivotal in mediating pleiotropic cellular responses to external stimuli. However, the potential role of GSK-3 inhibition in any given response is complicated by the fact that stimuli often initiate additional signalling pathways to the one that affects GSK-3 activity. Therefore, in order to more definitively implicate GSK-3 inhibition in a response, it is necessary to selectively inhibit this kinase and assess whether this alone is sufficient to induce the response.

Three isoforms of GSK3 are particularly relevant to the present invention, namely GSK-3α, GSK-3β and/or GSK-3β2, with GSK-3β being most relevant. Inhibitors of these enzymes and in particular, inhibitors of GSK-3β, are particularly preferred embodiments according to the present invention.

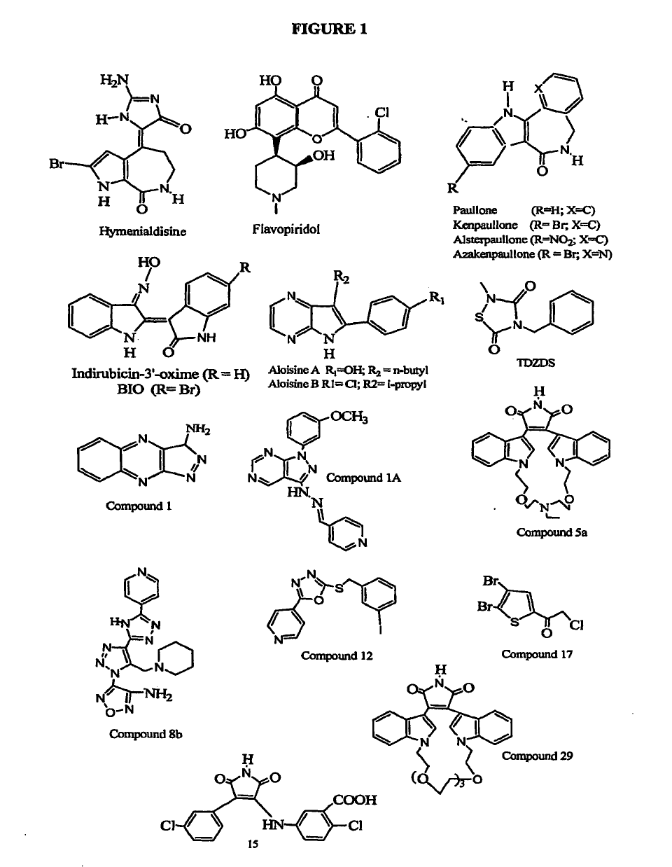

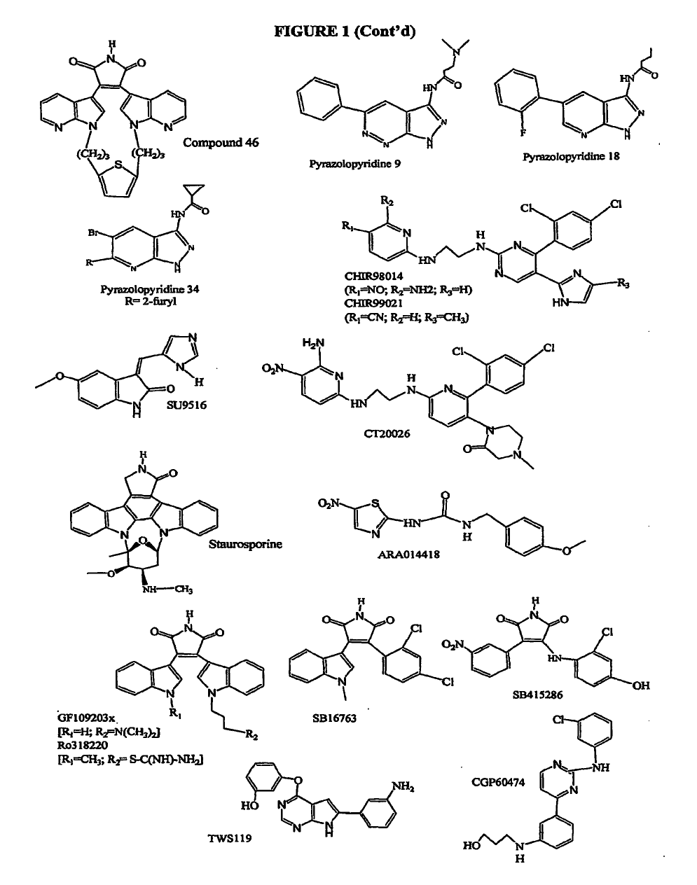

The term “GSK3 inhibitor” is used to describe one or more compounds which inhibits one or more (generally, all to a greater or lesser degree) of GSK-3α, GSK-3β and/or GSK-3β2, preferably GSK-3β. Preferred GSK3 inhibitors for use in the present invention are set forth in attached FIG. 1 and include, for example, pyrroloazepines, such as hymenialdisine; flavones, such as flavopiridol; benzazepinones such as kenpaullone, alsterpaullone and azakenpaullone; bis-indoles, such as indirubin-3′-Oxime, 6-Bromoindirubin-3′-oxime (BIO) and 6-Bromoindirubin-3′-acetoxime; pyrrolopyrazines, such as Aloisine A and Aloisine B; thiadiazolidinones, including TDZDB; pyridyloxadiazole, such as compound 12 of FIG. 1, pyrazolopyridines, such as pyrazolopyridine 18 and pyrazolopyridine 34 of FIG. 1, pyrazolopyridazine, such as pyrazolopyridine 9 of FIG. 1; aminopyrimidine, such as CHIR98014 and CHIR99021 (CT99021); aminopyridine, such as CT20026; pyrazoloquinoxalines, such as compound 1 of FIG. 1; oxindoles (Indolinone), such as SU9516; thiazoles, such as ARA014418; bisindolylmaleimides, such as staurosporine, compound 5a, GF109203x (bisindolylmaleimide I) and Ro318220 (bisindolylmaleimide IX); azaindolylmaleimide, such as compound 29 and compound 46 of FIG. 1; arylindolemaleimides, such as SB216763; anilinomaleimides, such as SB415286; anilinoarylmaleimides, such as compound 15, phenylaminopyrimidines, such as CGP60474; triazoles, such as compound 8b (FIG. 1); pyrrolopyrimidines, such as TWS119; pyrazolopyrimidines, such as compound 1A (FIG. 1); chloromethylthienylketones, such as compound 17 (FIG. 1). Of these compounds, SB216763 and SB415286 are preferred.

Additional GSK3 inhibitor compounds which may be used in the present invention include the 2-arylaminopyrimidine compounds which are described and set forth in United States patent application publication US 2004/0106574, Jun. 3, 2004 and the heteroarylamine compounds (GSK3β inhibitors) set forth in United patent application publication US2005/0004125, Jan. 6, 2005, both of which references are incorporated by reference in their entirety herein. Additional references include, for example, U.S. Pat. No. 7,045,519 to Nuss, et al., U.S. Pat. Nos. 7,053,097; 7,037,918; 6,989,382; 6,960,600; 6,949,547; 6,872,737; 6,800,632; 6,780,625; 6,608,063; 6,489,344; 6,479,490; 6,441,053; 6,417,085; 6,153,618 and 6,057,147, which are also directed to GSK3 inhibitors, and are incorporated by reference in their entirety herein.

Such GSK3 inhibitor compounds include those of United States patent application publication no. US 2004/0106574, Jun. 3, 2004, of the general structure:

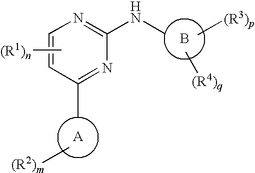

wherein:
Ring A is imidazo[1,2a]pyrid-3-yl or pyrazolo[2,3a]pyrid-3-yl;
R² is attached to a ring carbon and is selected from halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₃₋₆cycloalkyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆-alkyl)carbamoyl, N,N—(C₁₋₆alkyl)₂carbamoyl, C₁₋₆alkylS(O)ₐ wherein a is 0, 1 or 2, C₁₋₆alkoxycarbonyl, N—(C₁₋₆alkyl)sulphamoyl, N,N—(C₁₋₆alkyl)₂sulphamoyl, phenyl, heterocyclic group, phenylthio or (heterocyclic group)thio; wherein any C₁₋₆alkyl, C₂₋₆alkenyl, C₂₋₆alkynyl, phenyl or heterocyclic group may be optionally substituted on carbon by one or more G; and wherein if said heterocyclic group contains an —NH— moiety that nitrogen may be optionally substituted by a group selected from Q;
m is 0, 1, 2, 3, 4 or 5; wherein the values of R² may be the same or different;
R¹ is halo, nitro, cyano, hydroxy, trifluoromethyl, trifluoromethoxy, amino, carboxy, carbamoyl, mercapto, sulphamoyl, C₁₋₃alkyl, C₂₋₃alkenyl, C₂₋₃alkynyl, C₁₋₃alkoxy, C₁₋₃alkanoyl, N—(C₁₋₃alkyl)amino, N,N—(C₁₋₂alkyl)₂amino, C₁₋₃alkanoylamino, N—(C₁₋₃alkyl)carbamoyl, N,N—(C₁₋₂alkyl)₂carbamoyl, C₁₋₃alkylS(O)ₐ wherein a is 0, 1 or 2, N—(C₁₋₃alkyl)sulphamoyl or N,N—(C₁₋₃alkyl)₂sulphamoyl; wherein any C₁₋₂alkyl, C₁₋₃-alkyl, C₂₋₃alkenyl or C₂₋₃alkynyl may be optionally substituted on carbon by one or more J;
n is 0, 1 or 2, wherein the values of R¹ may be the same or different;
Ring B is phenyl or phenyl fused to a C₅₋₇cycloalkyl ring;
R³ is halo, nitro, cyano, hydroxy, amino, carboxy, carbamoyl, mercapto, sulphamoyl, C₁₋₆alkyl, C₂₋₆alkenyl or C₂₋₆alkynyl, C₁₋₆alkoxy;
p is 0, 1, 2, 3 or 4; wherein the values of R³ may be the same or different,
R⁴ is a group A-E-; wherein
A is selected from hydrogen, C₁₋₆alkyl, phenyl, a heterocyclic group, C₃₋₈cycloalkyl, phenylC₁₋₆alkyl, (heterocyclic group) C₁₋₆alkyl or C₃₋₈cycloalkylC₁₋₆cycloalkyl; which C₁₋₆alkyl, phenyl, a heterocyclic group, C₃₋₈cycloalkyl, phenylC₁₋₆alkyl, (heterocyclic group) C₁₋₆alkyl or C₃₋₈cycloalkylC₁₋₆cycloalkyl may be optionally substituted on carbon by one or more D; and wherein if said heterocyclic group contains an —NH— moiety that nitrogen may be optionally substituted by a group selected from R;
E is a direct bond or —O—, —C(O)—, —OC(O)—, —C(O)O—, —N(R^{a})C(O)—, —C(O)N(R^{a})—, —N(R^{a})—, —S(O)ᵣ—, —SO₂N(R^{a})— or —N(R^{a})SO₂—; wherein R^{a} is hydrogen or C₁₋₆alkyl optionally substituted by one or more D and r is 0, 1 or 2;
D is independently selected from oxo, halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₁alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0, 1 or 2, C₁₋₆alkoxycarbonyl, C₁₋₆alkoxycarbonylamino, benzyloxycarbonylamino, N—(C₁₋₆alkyl)sulphamoyl and N,N—(C₁₋₆alkyl)₂sulphamoyl; wherein any C₁₋₆alkyl, C₂₋₆alkenyl, C₂₋₆alkynyl or phenyl may be optionally substituted on carbon by one or more K;
q is 0, 1 or 2; wherein the values of R⁴ may be the same or different; and wherein p+q<=5;
G, J and K are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl; N,N-dimethylsulphamoyl, N,N-diethylsulphamoyl or N-methyl-N-ethylsulphamoyl; and
Q and R are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl; as a free base or a pharmaceutically acceptable salt thereof.
wherein:
Ring A is imidazo[1,2a]pyrid-3-yl or pyrazolo[2,3a]pyrid-3-yl;
R² is attached to a ring carbon and is selected from halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₃₋₆cycloalkyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0, 1 or 2, C₁₋₆alkoxycarbonyl, N—(C₁₋₆alkyl)sulphamoyl, N,N—(C₁₋₆alkyl)₂sulphamoyl, phenyl, heterocyclic group, phenylthio or (heterocyclic group)thio; wherein any C₁₋₆alkyl, C₂₋₆alkenyl, C₂₋₆alkynyl, phenyl or heterocyclic group may be optionally substituted on carbon by one or more G; and wherein if said heterocyclic group contains an —NH— moiety that nitrogen may be optionally substituted by a group selected from Q;
m is 0, 1, 2, 3, 4 or 5; wherein the values of R² may be the same or different;
R¹ is halo, nitro, cyano, hydroxy, trifluoromethyl, trifluoromethoxy, amino, carboxy, carbamoyl, mercapto, sulphamoyl, C₁₋₃alkyl, C₂₋₃alkenyl, C₂₋₃alkynyl, C₁₋₃alkoxy, C₁₋₃alkanoyl, N—(C₁₋₃alkyl)amino, N,N—(C₁₋₂alkyl)₂amino, C₁₋₃alkanoylamino, N—(C₁₋₃alkyl)carbamoyl, N,N—(C₁₋₂alkyl)₂carbamoyl, C₁₋₃alkylS(O)ₐ wherein a is 0, 1 or 2, N—(C₁₋₃alkyl)sulphamoyl or N,N—(C₁₋₃alkyl)₂sulphamoyl; wherein any C₁₋₂alkyl, C₁₋₃alkyl, C₂₋₃alkenyl or C₂₋₃alkynyl may be optionally substituted on carbon by one or more J;
n is 0, 1 or 2, wherein the values of R¹ may be the same or different;
Ring B is phenyl or phenyl fused to a C₅₋₇cycloalkyl ring;
R³ is halo, nitro, cyano, hydroxy, amino, carboxy, carbamoyl, mercapto, sulphamoyl, C₁₋₆alkyl, C₂₋₆alkenyl or C₂₋₆alkynyl, C₁₋₆alkoxy;
p is 0, 1, 2, 3 or 4; wherein the values of R³ may be the same or different;
R⁴ is a group A-E-; wherein
A is selected from hydrogen, C₁₋₆alkyl, phenyl, a heterocyclic group, C₃₋₈cycloalkyl, phenylC₁₋₆alkyl, (heterocyclic group) C₁₋₆alkyl or C₃₋₈-cycloalkylC₁₋₆cycloalkyl; which C₁₋₆alkyl, phenyl, a heterocyclic group, C₃₋₈cycloalkyl, phenylC₁₋₆alkyl, (heterocyclic group) C₁₋₆alkyl or C₃₋₈cycloalkylC₁₋₆cycloalkyl may be optionally substituted on carbon by one or more D; and wherein if said heterocyclic group contains an —NH— moiety that nitrogen may be optionally substituted by a group selected from R;
E is a direct bond or —O—, —C(O)—, —OC(O)—, —C(O)O—, —N(R^{a})C(O)—, —C(O)N(R^{a})—, —N(R^{a})—, —S(O)ᵣ—, —SO₂N(R^{a})— or —N(R^{a})SO₂—; wherein R^{a} is hydrogen or C₁₋₆alkyl optionally substituted by one or more D and r is 0, 1 or 2;
D is independently selected from oxo, halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₁alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0, 1 or 2, C₁₋₆alkoxycarbonyl, C₁₋₆alkoxycarbonylamino, benzyloxycarbonylamino, N—(C₁₋₆alkyl)sulphamoyl and N,N—(C₁₋₆alkyl)₂sulphamoyl; wherein any C₁₋₆alkyl, C₂₋₆alkenyl, C₂₋₆alkynyl or phenyl may be optionally substituted on carbon by one or more K;
q is 0, 1 or 2; wherein the values of R⁴ may be the same or different; and wherein p+q<=5;
G, J and K are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; and
Q and R are independently selected from C₁₋₄alkyl, C₁₋₄alkanoyl, C₁₋₄alkylsulphonyl, C₁₋₄-alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl; as a free base or a pharmaceutically acceptable salt thereof.

More specific compounds include:
2-(4-Fluoro-3-methylanilino)-4-(imidazo[1,2a]pyrid-3-yl)pyrimidine;
2-(4-Cyanoanilino)-4-(imidazo[1,2a]pyrid-3-yl)pyrimidine;
2-(4-Chloroanilino)-4-(imidazo[1,2a]pyrid-3-yl)pyrimidine;
2-Anilino-4-(2-methylimidazo[1,2a]pyrid-3-yl)pyrimidine;
2-[4-(Pyrimid-2-ylaminosulphonyl)anilino]-4-(imidazo[1,2a]pyrid-3-yl)pyrimidine,
2-(4-Carbamoylamino)-4-(imidazo[1,2a]pyrid-3-yl)pyrimidine,
2-(3-Cyanoanilino)-4-(imidazo[1,2a]pyrid-3-yl)pyrimidine,
2-(3,5-Difluoroanilino)-4-(imidazo[1,2a]pyrid-3-yl)pyrimidine,
2-(3-Chloroanilino)-4-(imidazo[1,2a]pyrid-3-yl)pyrimidine,
2-[4-N,N-Dimethyl-carbamoyl)anilino]4-(imidazo[1,2a]pyrid-3-yl)pyrimidine,
2-(4-Mesylanilino)-4-(imidazo[1,2a]pyrid-3-yl)pyrimidine and
2-(3-Sulphamoylanilino)-4-(imidazo[1,2a]pyrid-3-yl)pyrimidine,
   as a free base or pharmaceutically acceptable salt thereof.

Other GSK3 inhibitors include compounds of United States patent application publication no. 2005/0004125, Jan. 6, 2005, according to the structure:

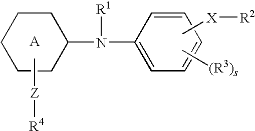

a N-oxide, a pharmaceutically acceptable addition salt, a quaternary amine and a stereochemically isomeric form thereof, wherein ring A is pyridyl, pyrimidinyl, pyrazinyl or pyridazinyl;
R¹ is hydrogen; aryl; formyl; C₁₋₆ alkylcarbonyl; C₁₋₆ alkyl; C₁₋₆alkyloxycarbonyl; C₁₋₆alkyl substituted with formyl, C₁₋₆alkylcarbonyl, C₁₋₆alkyloxycarbonyl, C₁₋₆alkylcarbonyloxy; C₁₋₆alkyloxyC₁₋₆alkylcarbonyl optionally substituted with C₁₋₆alkyloxycarbonyl;
X is —NR¹—; —NH—NH—; —N═N—; —O—; —C(═O)—; —C(═S)—; —O—C(═O)—; —C(═O)—O—; —O—C(═O)—C₁₋₆alkyl-; —C(═O)—O—C₁₋₆alkyl-; —O—C₁₋₆alkyl-C(═O)—; —C(═O)—C₁₋₆alkyl-O—; —O—C(═O)—NR¹—; —NR¹—C(═O)—O—; —O—C(═O)—C(═O)—; —C(═O)—NR¹—, —NR¹—C(═O)—; —C(═S)—NR¹—, —NR¹—C(═S)—; —NR¹—C(═O)—NR¹—; —NR¹—C(═S)—NR¹—; —NR¹—S(═O)—NR¹—; —NR¹—S(═O)₂—NR¹—; —C₁₋₆alkyl-C(═O)—NR¹—; —O—C₁₋₆alkyl-C(═O)—NR¹—; —C₁₋₆alkyl-O—C(═O)—NR¹—; —C₁₋₆alkyl-; —O—C₁₋₆alkyl-; —C₁₋₆alkyl-O—; —NR¹—C₁₋₆alkyl-; —C₁₋₆alkyl-NR¹—; —NR¹—C₁₋₆alkyl-NR¹—; —NR¹—C₁₋₆alkyl-C₃₋₇cycloalkyl-; —C₂₋₆alkenyl-; —C₂₋₆alkynyl-; —O—C₂₋₆alkenyl-; —C₂₋₆alkenyl-O—; —NR¹—C₂₋₆alkenyl-; —C₂₋₆alkenyl-NR¹—; —NR₁—C₂₋₆alkenyl-NR¹—; —NR¹—C₂₋₆alkenyl-C₃₋₇cycloalkyl-; —O—C₂₋₆alkynyl-; —C₂₋₆alkynyl-O—; —NR¹—C₂₋₆alkynyl-; —C₂₋₆alkynyl-NR¹—; —NR¹—C₂₋₆alkynyl-NR¹—; —NR¹—C₂₋₆alkynyl-C₃₋₇cycloalkyl-; —O—C₁₋₆alkyl-O—; —O—C₂₋₆alkenyl-O—; —O—C₂₋₆alkynyl-O—; —CHOH—; —S—; —S(═O)—; —S(═O)₂—; —S(═O)—NR¹—; —S(═O)₂—NR¹—; —NR¹—S(═O)—; —NR¹—S(═O)₂—; —S—C₁₋₆alkyl-; —C₁₋₆ alkyl-S—; —S—C₂₋₆alkenyl-; —C₂₋₆alkenyl-S—; —S—C₂₋₆alkynyl-; —C₂₋₆alkynyl-S—; —O—C₁₋₆alkyl-S(═O)₂— or a direct bond;
Z is a direct bond, C₁₋₆alkanediyl, C₂₋₆alkenediyl, C₂₋₆alkenediyl; —O—; —O—C₁₋₆alkyl-; —S—; —C(═O)—; —C(═O)—O—; —O—C(═O)—; —C(═S)—; —S(═O)—; —S(═O)₂—; —NR¹—; —NR¹—C₁₋₆alkyl-; —NR¹—C(═O)—; —O—C(═O)—NR¹—; —NR¹C(═O)—; —O—; —NR¹—C(═S)—; —S(═O)—NR¹—; —S(═O)₂—NR¹—, —NR¹—S(═O)—; —NR¹—S(═O)₂—; —NR¹—(C═O)—NR¹—; —NR¹—C(═S)—NR¹—; —NR¹—S(═O)—NR¹—; —NR¹—S(═O)₂—NR¹—;
R² is hydrogen, C₁₋₁₀alkyl, C₂₋₁₀alkenyl, C₂₋₁₀alkynyl, R²⁰, each of said groups representing R² may optionally be substituted where possible with one or more substituents each independently being selected from ═S; ═O; R¹⁵; hydroxy; halo; nitro; cyano; R¹⁵—O—; SH; R¹⁵—S—; formyl; carboxyl; R¹⁵—C(═O)—; R¹⁵—O—C(═O)—; R¹⁵—C(═O)—O—; R¹⁵—O—C(═O)—O—; —SO₃H; R¹⁵—S(═O)—; R¹⁵—S(═O)₂—; R⁵R⁶N; R⁵R⁶N—C₁₋₆alkyl; R⁵R⁶N—C₃₋₇cycloakyl; R⁵R⁶N—C₁₋₆alkyloxy; R⁵R⁶N—C(═O)—; R⁵R⁶N—C(═S)—; R⁵R⁶N—C(═O)—NH—; R⁵R⁶N—C(═S)—NH—; R⁵R⁶N—S(═O)ₙ—; R⁵R⁶N—S(═O)ₙ—NH—; R¹⁵—C(═S)—; R¹⁵—C(═O)—NH—; R¹⁵—O—C(═O)—NH—; R¹⁵—S(═O)ₙ—NH—; R¹⁵—O—S(═O), —NH—; R¹⁵—C(═S)—NH—; R¹⁵—O—C(═S)—NH—; R¹⁷R¹⁸N—Y₁ₐ—; R¹⁷R¹⁸N—Y₂—NR⁶—Y₁—; R⁵—Y₂—NR¹⁹—Y₁—; H—Y₂—NR¹⁹—Y₁—;
R³ is hydrogen; hydroxy; halo; C₁₋₆alkyl; C₁₋₆alkyl substituted with cyano, hydroxy or —C(═O)R⁷; C₂₋₆alkenyl; C₂₋₆alkenyl substituted with one or more halogen atoms or cyano; C₂₋₆alkynyl; C₂₋₆alkynyl substituted with one or more halogen atoms or cyano; C₁₋₆alkyloxy; C₁₋₆alkylthio; C₁₋₆alkyloxycarbonyl; C₁₋₆alkylcarbonyloxy; carboxyl; cyano; nitro; amino; mono- or di(C₁₋₆alkyl)amino; polyhaloC₁₋₆alkyl; polyhaloC₁₋₆alkyloxy; polyhaloC₁₋₆alkylthio; R²¹; R²¹—C₁₋₆alkyl; R²—O—; R²—S—; R²¹—C(═O)—; R²¹—S(═O)ₙ—; R⁷—S(═O)ₚ—; R⁷—S(═O)ₚ—NH—; R²—S(═O)ₚ—NH—; R⁷—C(═O)—; —NHC(═O)H; —C(═O)NHNH₂; R⁷—C(═O)—NH—; R²¹—C(═O)—NH—; —C(—NH)R⁷; —C(—NH)R²¹;
R⁴ is a monocyclic, bicyclic or tricyclic saturated heterocycle; a monocyclic, bicyclic or tricyclic partially saturated heterocycle or a monocyclic, bicyclic or tricyclic aromatic heterocycle, each of said heterocycles optionally being substituted where possible with one or more substituents each independently being selected from ═S; ═O; R¹⁵; hydroxy; halo; nitro; cyano; R¹⁵—O—; SH; R¹⁵—S—; formyl; carboxyl; R¹⁵—C(═O)—; R¹⁵—O—C(═)—; R¹⁵—C(═O)—; —O—; R¹⁵—O—C(═O)—O—; —SO₃H; R¹⁵—S(—O)—; R¹⁵—S(═O)₂—; R⁵R⁶N; R⁵R⁶NC₆alkyl; R⁵R⁶NC₃₋₇cycloalkyl; R⁵R⁶NC₁₋₆alkyloxy; R⁵R⁶N—C(═O)—; R⁵R⁶N—C(═S)—; R⁵R⁶N—C(═O)—NH—; R⁵R⁶N—C(═S)—NH—; R⁵R⁶N—S(═O)ₙ—; R⁵R⁶N—S(═O)ₙ, —NH—; R¹⁵—C(═S)—; R¹⁵—C(═O)—NH—; R¹⁵—O(═O)—NH—; R¹⁵—S(═O)ₙ, —NH—; R⁵—O—S(═O), —NH—; R¹⁵C(═S)—NH—; R¹⁵—O—C(═S)—NH—; R¹⁷R¹⁸N—Y₁ₐ—; R¹⁷R¹⁸N—Y₂—NR¹⁶—Y₁—; R⁵—Y₂—NR¹⁹—Y₁—; H—Y₂—NR¹⁹—Y₁—;
R⁵ and R⁶ each independently are hydrogen, R⁸, —Y₁—NR⁹—Y₂—NR¹⁰R¹¹, —Y₁—NR⁹—Y₁—R⁸, —Y₁—NR⁹R¹⁰, or
R⁵ and R⁶ may together with the nitrogen to which they are attached form a saturated or partially saturated monocyclic 3 to 8 membered heterocycle or an aromatic 4 to 8 membered monocyclic heterocycle, each of said heterocycles may optionally be substituted with one or more substituents selected from R¹², R¹³ and R¹⁴, or each of said heterocycles may optionally be fused with a benzene ring, said benzene ring being optionally substituted with one or more substituents selected from R¹², R¹³ and R¹⁴;
R⁷ is C₁₋₆alkyl, C₁₋₆alkyloxy, amino, mono- or di(C₁₋₆alkyl)amino or polyhaloC₁₋₆alkyl;
R⁸ is C₁₋₆alkyl; C₂₋₆alkenyl; C₂₋₆alkynyl; a monocyclic, bicyclic or tricyclic saturated carbocycle; a monocyclic, bicyclic or tricyclic partially saturated carbocycle; a monocyclic, bicyclic or tricyclic aromatic carbocycle; a monocyclic, bicyclic or tricyclic saturated heterocycle; a monocyclic, bicyclic or tricyclic partially saturated heterocycle; a monocyclic, bicyclic or tricyclic aromatic heterocycle; C₁₋₆alkyl substituted with a monocyclic, bicyclic or tricyclic saturated carbocycle or with a monocyclic, bicyclic or tricyclic partially saturated carbocycle or with a monocyclic, bicyclic or tricyclic aromatic carbocycle or with a monocyclic, bicyclic or tricyclic saturated heterocycle or with a monocyclic, bicyclic or tricyclic partially saturated heterocycle or with a monocyclic, bicyclic or tricyclic aromatic heterocycle; each of said groups representing R⁸ may optionally be substituted with one or more substituents selected from R¹², R¹³ and R¹⁴;
R⁹, R¹⁰ and R¹¹ each independently are hydrogen or R⁸, or
any two of R⁹, R¹⁰ and R¹¹ may together be C₁₋₆alkanediyl or C₂₋₆alkenediyl thereby forming a saturated or partially saturated monocyclic 3 to 8 membered heterocycle or an aromatic 4 to 8 membered monocyclic heterocycle together with the nitrogen atoms to which they are attached, each of said heterocycles may optionally be substituted with one or more substituents selected from R¹², R¹³ and R¹⁴;

R¹², R¹³ and R¹⁴ each independently are hydrogen; R¹⁵; hydroxy, halo; nitro; cyano; R¹⁵—O—; SH; R¹⁵—S—; formyl; carboxyl; R¹⁵—C(═O)—; R¹⁵—O—C(═O)—; R¹⁵—C(═O)—O—; R¹⁵—O—C(═O)—O—; —SO₃H; R¹⁵—S(═O)—; R¹⁵—S(═O)₂—; R¹⁵R¹⁶N—S(═O)—; R¹⁵R¹⁶N—S(═O)₂—; R¹⁷R¹⁸N—Y₁—; R¹⁷R¹⁸N—Y₂—NR¹⁶—Y₁—; R¹⁵—Y₂—NR¹⁹—Y₁—; H—Y₂—NR¹⁹—Y₁—; oxo, or

any two of R¹², R¹³ and R¹⁴ may together be C₁₋₆alkanediyl or C₂₋₆alkenediyl thereby forming a saturated or partially saturated monocyclic 3 to 8 membered carbo- or heterocycle or an aromatic 4 to 8 membered monocyclic carbo- or heterocycle together with the atoms to which they are attached, or any two of R¹², R¹³ and R¹⁴ may together be —O—(CH₂)ᵣ—O— thereby forming a saturated, partially saturated or aromatic monocyclic 4 to 8 membered carbo- or heterocycle together with the atoms to which they are attached;
R¹⁵ is C₁₋₆alkyl C₂₋₆alkenyl, C₂₋₆alkynyl, a monocyclic, bicyclic or tricyclic saturated carbocycle; a monocyclic, bicyclic or tricyclic partially saturated carbocycle; a monocyclic, bicyclic or tricyclic aromatic carbocycle; a monocyclic, bicyclic or tricyclic saturated heterocycle; a monocyclic, bicyclic or tricyclic partially saturated heterocycle; a monocyclic, bicyclic or tricyclic aromatic heterocycle; C₁₋₆alkyl substituted with a monocyclic, bicyclic or tricyclic saturated carbocycle or with a monocyclic, bicyclic or tricyclic partially saturated carbocycle or with a monocyclic, bicyclic or tricyclic aromatic carbocycle or with a monocyclic, bicyclic or tricyclic saturated heterocycle or with a monocyclic, bicyclic or tricyclic partially saturated heterocycle or with a monocyclic, bicyclic or tricyclic aromatic heterocycle; each of said substituents representing R¹⁵ may optionally be substituted with one or more substituents selected from R¹², R¹³ and R¹⁴; or each of said carbocycles or heterocycles may optionally be fused with a benzene ring, said benzene ring being optionally substituted with one or more substituents selected from R¹², R¹³ and R¹⁴;
R¹⁶, R¹⁷, R¹⁸ and R¹⁹ each independently are hydrogen or R¹⁵, or
R¹⁷ and R¹⁸, or R¹⁵ and R¹⁹ may together be C₁₋₆alkanediyl or C₂₋₆alkenediyl thereby forming a saturated or partially saturated monocyclic 3 to 8 membered heterocycle or an aromatic 4 to 8 membered monocyclic heterocycle, each of said heterocycles may optionally be substituted with one or more substituents selected from R¹², R¹³ and R¹⁴; or
R¹⁷ and R¹⁸ together with R¹⁶ may be C.sub.1-6alkanediyl or C₂₋₆alkenediyl thereby forming a saturated or partially saturated monocyclic 3 to 8 membered heterocycle or an aromatic 4 to 8 membered monocyclic heterocycle together with the nitrogen atoms to which they are attached, each of said heterocycles may optionally be substituted with one or more substituents selected from R¹², R¹³ and R¹⁴;
R²⁰ is a monocyclic, bicyclic or tricyclic saturated carbocycle; a monocyclic, bicyclic or tricyclic partially saturated carbocycle; a monocyclic, bicyclic or tricyclic aromatic carbocycle; a monocyclic, bicyclic or tricyclic saturated heterocycle; a monocyclic, bicyclic or tricyclic partially saturated heterocycle; a monocyclic, bicyclic or tricyclic aromatic heterocycle;
R²¹ is a monocyclic, bicyclic or tricyclic saturated carbocycle; a monocyclic, bicyclic or tricyclic partially saturated carbocycle; a monocyclic, bicyclic or tricyclic aromatic carbocycle; a monocyclic, bicyclic or tricyclic saturated heterocycle; a monocyclic, bicyclic or tricyclic partially saturated heterocycle; a monocyclic, bicyclic or tricyclic aromatic heterocycle, each of said carbocycles or heterocycles representing R²¹ may optionally be substituted with one or more substituents selected from R¹², R¹³ and R¹⁴;
Y₁ₐ is —Y₃—S(═O)—Y₄—; —Y₃—S(═O)₂—Y₄—, —Y₃—C(═O)—Y₄—, —Y₃—C(═S)—Y₄—, Y₃—O—Y₄—, —Y₃—S—Y₄—, —Y₃—O—C(═O)—Y₄— or —Y₃—C(═O)—O—Y₄—;
Y₁ or Y₂ each independently are a direct bond, —Y₃—S(═O)—Y₄—; —Y₃—S(═O)₂—Y₄—, —Y₃—C(═O)Y₄—, —Y₃—C(═S)—Y₄—, —Y₃—O—Y₄—, —Y₃—S—Y₄—, —Y₃—O—C(═O)—Y₄— or —Y₃—C(═O)—O—Y₄—;
Y₃ or Y₄ each independently are a direct bond, C₁₋₆alkanediyl, C₂₋₆alkenediyl or C₂₋₆alkynediyl;
n is 1 or 2;
m is 1 or 2;
p is 1 or 2;
r is 1 to 5;
s is 1 to 3;
aryl is phenyl or phenyl substituted with one, two, three, four or five substituents each independently selected from halo, C₁₋₆alkyl, C₃₋₇cycloalkyl, C₁₋₆alkyloxy, cyano, nitro, polyhalo C₁₋₆alkyl and polyhalo C₁₋₆alkyloxy; provided that —X—R² and/or R³ is other than hydrogen.

More specific compounds include:
N²-(1H-indazol-5-yl)-N⁴-(2,4,6-trimethylphenyl)-2,4-pyrimidinediamine;
4-[[4-(1-methyl-1H-imidazol-2-yl)-2-pyrimidinyl]amino]-2-(phenylmethoxy)-benzonitrile;
4-[[4-(1-methyl-1H-imidazol-2-yl)-2-pyrimidinyl]amino]-benzonitrile;
a N-oxide, a pharmaceutically acceptable addition salt, a quaternary amine OR a stereochemically isomeric form thereof.

Other preferred compounds may include:
N-(6-morpholinyl-4-yl-pyridin-3-yl)-N-4-(2,4,6-trimethyl-phen-yl)-2,4-pyrimidinediamine;
N²-(3H-benzimidazol-5-yl)-N⁴-(2,4,6-trimethyl-phenyl)-2,4-pyrimidinediamine;
N²-(1H-indazol-6-yl)-N⁴-(2,4,6-trimethyl-phenyl)-2,4-pyrimidinediamine;
N²-(5-bromo-pyridin-2-yl)-N-4-(2,4,6-trimethyl-phenyl)-2,4-pyrimidinediamine;
N²-(6-methoxy-pyridin-3-yl)-N⁴-(2,4,6-trimethyl-phenyl)-2,4-pyrimidinediamine;
N²-benzothiazol-6-yl-N-4-(2,4,6-trimethyl-phenyl)-2,4-pyrimidinediamine;
N²-(1H-indazol-5-yl)-N⁴-(2,4,6-trimethyl-phenyl)-2,4-pyrimidinediamine;
N²-(1H-benzotriazol-5-yl)-N-4-(2,4,6-trimethyl-phenyl)-2,4-pyrimidinediamine;
N²-benzo[1,3]dioxol-5-yl-N⁴-(2,4,6-trimethyl-phenyl)-2,4-pyrimidinediamine;
N²-(6-chloro-pyridin-3-yl)-N⁴-(2,4,6-trimethyl-phenyl)-2,4-pyrimidinediamine
N²-(1H-indol-5-yl)-N⁴-(2,4,6-trimethyl-phenyl)-2,4-pyrimidinediamine;
N²-quinolin-6-yl-N-4-(2,4,6-trimethyl-phenyl)-2,4-pyrimidinediamine;
4-[4-[(benzo[1,3]dioxol-5-ylmethyl)-amino]-pyrimidin-2-ylamino]-benzonitrile;
4-[4-[(quinolin-3-methyl)-amino]-pyrimidin-2-ylamino]-benzonitrile;
4-[4-[(furan-2-ylmethyl)-amino]-pyrimidin-2-ylamino]-benzonitrile;
4-[4-[(thiophen-2-ylmethyl)-amino]-pyrimidin-2-ylamino]-benzonitrile;
a N-oxide, a pharmaceutically acceptable addition salt, a quaternary amine and a stereochemically isomeric form thereof.

A “pharmaceutically acceptable salt” of a compound used in the present invention generally refers to pharmaceutically acceptable salts form of a compound which can form a salt, because of the existence of for example, amine groups, carboxylic acid groups or other groups which can be ionized in a sample acid-base reaction. A pharmaceutically acceptable salt of an amine compound, such as those contemplated in the current invention, include, for example, ammonium salts having as counterion an inorganic anion such as chloride, bromide, iodide, sulfate, sulfite, nitrate, nitrite, phosphate, and the like, or an organic anion such as acetate, malonate, pyruvate, propionate, fumarate, cinnamate, tosylate, and the like. Certain compounds according to the present invention which have carboxylic acid groups or other acidic groups which may form pharmaceutically acceptable salts, for example, as carboxylate salts (potassium, sodium, magnesium, zinc, ammonium, etc.) are also contemplated by the present invention.

Aspects of the present invention include compounds which have been described in detail hereinabove or to pharmaceutical compositions which comprise an effective amount of one or more compounds according to the present invention, optionally in combination with a pharmaceutically acceptable carrier, additive or excipient.

The term “effective” shall mean, within context, an amount of a compound, composition or component and for a duration of time (which may vary greatly depending upon the disease state, condition or manifestation to be treated or to have a reduced likelihood of occurring) which produces an intended effect within the context of the use of the compound, composition or component. In instances where more than one compound is administered (coadministration) or a component is used, that compound or component is used in an effective amount to produce a desired or intended effect, very often, a favorable therapeutic outcome.

The term “E-cadherin/β-catenin pathway” in dendritic cells is used to refer to a pathway for dendritic cell maturation in the present invention, resulting in mature dendritic cells which exhibit immune T-cell tolerance. We can be sure that the β-catenin pathway is activated by measuring the increased level of activity (transcription) of genes well known to be under the transcriptional control of β-catenin and its associated transcriptional activators TCF/LEF. This may be measured by looking for the enhanced expression of selected target genes themselves or by monitoring the output of artificial “reporter genes” introduced into cells for the purpose of demonstrating when β-catenin-dependent activation takes place.

The data which are presented herein evidence that the dendritic cell (DC) markers which are associated with the induction of tolerance (“tolerogenic DC”), are distinguished from “immunogenic” DCs by a dramatically reduced ability to secrete inflammatory cytokines (eg., IL6, IL12, IL1β, as otherwise disclosed herein). Although both tolerogenic and immunogenic DCs express surface costimulatory molecules (CD80, CD86) required for T cell activation, only immunogenic DCs secrete appreciable quantities of cytokines (i.e., cytokine secretion is much reduced in tolerogenic DC's, indicating that cytokine secretion is required to promulgate an effective immune response. In general, tolerogenic DC's excrete cytokines in amounts generally less than 80% of immunogenic DC's, more preferably less than 50% of immunogenic DC's, in many cases less than 20% of immunogenic DC's. In certain instances, tolerogenic DC's will not secrete appreciable (ie., measurable concentrations or quantities of cytokines).

The term “mature dendritic cell” is used throughout the specification to refer to a dendritic cell which has been exposed to a GSK3 inhibitor to activate the E-cadherin/β-catenin pathway in those dendritic cells and produce “mature” dendritic cells which exhibit a T cell response associated with the induction or maintenance of T cell tolerance in said patient. The type of mature dendritic cells produced using the methods of the present invention exhibit many of the features of dendritic cells matured by microbial stimuli, such as an increase in major histocompatibility (MHC) type II products, costimulatory molecules (e.g., CD80, CD86), and the upregulation of chemokine receptors required for dendritic cell migration. The mature cells are distinguished from other dendritic cells by a markedly reduced (about 80% or less, about 60% or less, about 50% or less, about 40% or less, about 30% or less, about 20% or less of typical dendritic cell production of one or more inflammatory or immunogenic cytokine) ability to produce inflammatory or immunogenic cytokines such as IL-1α, IL-1β, IL-2, IL-4, IL-5, IL-6, IL-10, IL-12, IL-13, IL-15, TNF-alpha, MCP1, CXCL8, RANTES (CCL5) and CCL22. Mature dendritic cells produced using GSK3 inhibitors according to the present invention exhibit cytokine (chemokine) profiles virtually identical to dendritic cells matured by cluster disruption, with the exception that IL1α and RANTES (CCL5) were significantly produced but in much lower levels than that produced by LPS or bacteria.

The term “immune tolerance” is used throughout the specification to refer to an immunological state in which an individual fails to mount an immune response to a particular foreign (immune dysfunction/dysregulation) or self antigen (autoimmune disease). It is characterized by a failure of T lymphocytes to produce cytokines that yield the classical hallmarks of inflammation. Instead, T lymphocyte responses should they occur at all are of the “regulatory” type, meaning they involve the production of cells that actively limit immune responsiveness. Since it is possible to induce tolerance to noxious environmental stimuli associated with allergy or inflammation, it then becomes possible to ameliorate a variety of chronic and acute inflammatory states such as asthma, inflammatory bowel disease, and rheumatoid arthritis, among numerous others, as otherwise set forth herein.

In the present invention, by providing for the inhibition of GSK3 in the presence of known or unknown autoimmune targets or exogenous and endogenous inflammatory targets, dendritic cells can be induced to help stimulate “tolerance” to such offending targets thus reducing pathogenic immune responses. For example, administration of an inhibitor to GSK3 intranasally could reduce the pulmonary responses to airborne environmental antigens that are the cause of asthma.

The term “autoimmune disease”, “disease associated with immune dysfunction/dysregulation” or “immune inflammatory disease” is used throughout the specification to refer to a pathogenic condition in which the patients immune system results in disease from a self antigen (autoimmunity) or a foreign antigen (immune dysfunction/dysregulation or immune inflammatory disease). Autoimmunity is present in everyone to some extent. It is usually harmless and probably a universal phenomenon of vertebrate life. However, autoimmunity can be the cause of a broad spectrum of human illnesses, known as autoimmune diseases. This concept of autoimmunity as the cause of human illness is relatively new, and it was not accepted into the mainstream of medical thinking until the 1950s and 1960s. Autoimmune diseases are, thus, defined when the progression from benign autoimmunity to pathogenic autoimmunity occurs. This progression is determined by both genetic influences and environmental triggers. The concept of autoimmunity as the actual cause of human illness (rather than a consequence or harmless accompaniment) can be used to establish criteria that define a disease as an autoimmune disease. By this approach, Rose and Bona (Immunology Today, 14: 426-430, 1993) have distinguished the evidence for an autoimmune etiology at three different levels: direct, indirect, and circumstantial.

Direct evidence requires transmissibility of the characteristic lesions of the disease from human to human, or human to animal. In the real world, such evidence is attainable at this time only for diseases mediated by autoantibody, since we do not yet have the means for reliably studying T lymphocyte-mediated autoimmune diseases by transfer to animals. Examples of autoimmune diseases that fulfill the criteria of direct evidence are idiopathic thrombocytopenic purpura (in which deliberate human experimentation in the early 1950s showed that the platelet destruction is directly caused by an autoantibody), Graves' disease and myasthenia gravis (in which there are temporary signs of disease in the infant due to transplacental transfer), pemphigus vulgaris and bullous pemphigoid (where the disease can be transmitted from humans to animals by autoantibody). Another, more feasible, way to demonstrate pathologic effect of autoantibody is to reproduce the functional defects characteristic of the disease in vitro. For example, inhibition of the fixation of vitamin B12 by intrinsic factor can be produced by autoantibodies from certain patients with pernicious anemia, and overproduction of thyroid hormones can be produced by autoantibodies from patients with Graves' disease.

Indirect evidence requires re-creation of the human disease in an animal model. The majority of autoimmune diseases fit in this category. For example, the autoimmune basis of systemic lupus erythematosus (SLE) is well accepted because of the availability of several genetically determined mouse models which, while not simulating lupus as seen in the clinic, do very closely replicate the serological features and some pathological features. Hashimoto's thyroiditis and multiple sclerosis can be reproduced by immunizing the animal with an antigen analogous to the putative autoantigen of the human disease. The development of animal models is increasing rapidly as methods of genetic and immunologic manipulation become commonplace. For example, knock-out mice have provided the best models of inflammatory bowel disease; neonatal thymectomy of mice can produce excellent analogs of human oophoritis and autoimmune gastritis. It is worth noting that animal models must be viewed with caution as being an analog rather than the exact copy of the human counterpart, because they invariably differ to some degree from the human disease.

When direct and indirect evidence to define an autoimmune disease are not available, investigators are left with circumstantial evidence, that is, with listing “markers” descriptive of autoimmune disease. Examples of these markers are:
positive family history for the same disease, or for other diseases known to be autoimmune;
presence in the same patient of other known autoimmune diseases;
presence of infiltrating mononuclear cells in the affected organ or tissue;
preferential usage of certain MHC class II allele
high serum levels of IgG autoantibodies
deposition of antigen-antibody complexes in the affected organ or tissue
improvement of symptom with the use of immunosuppressive drugs (such as corticosteroids)

Autoimmune diseases exhibit a broad spectrum. Autoimmune diseases can strike any part of the body, and thus symptoms vary widely and diagnosis and treatment are often difficult. The broad spectrum of autoimmune diseases or diseases of immune dysfunction/dysregulation includes asthma, multiple sclerosis and the severe type 1 diabetes mellitus. Some autoimmune diseases such as lupus (SLE) and pemphigus can be life threatening unless properly diagnosed and treated. Chromic autoimmune disorders like rheumatoid arthritis cripple the patient and also create heavy burdens on patients' families. Some types of uveitis may cause blindness. Diseases such as scleroderma require skillful, lifelong treatment. Still other autoimmune diseases, including Graves' disease and chronic thyroiditis, can be successfully treated if correctly diagnosed, but they are frequently missed because of their subtle onset.

Autoimmune diseases or diseases which are characterized as involving immune dysfunction or dysregulation (immune inflammatory disease), which may be treated by the present invention include systemic lupus erythematosis (SLE), diabetes mellitus (type I), asthma, Grave's disease, arthritis, including rheumatoid arthritis and osteoarthritis, pernicious anemia, and multiple sclerosis, among numerous others. Numerous autoimmune diseases may be treated using the method of the present invention including autoimmune blood diseases, including pernicious anemia, autoimmune hemolytic anemia, aplastic anemia, idiopathic thrombocytopenic purpura, ankylosing spondylitis; autoimmune diseases of the musculature including polymyositis and dermatomyositis, autoimmune diseases of the ear including autoimmune hearing loss and Meniere's syndrome, autoimmune eye diseases, including Mooren's disease, Reiter's syndrome and Vogt-Koyanagi-Harada disease, autoimmune diseases of the kidney including glomerulonephritis and IgA nephropathy; diabetes mellitus (type I); autoimmune skin diseases including pemphigus (autoimmune bullous diseases), such as pemphigus vulgaris, pemphigus foliaceus, pemphigus erythematosus, bullous pemphigoid, vitiligo, epidermolysis bullosa acquisita, and alopecia areata; cardiovascular autoimmune diseases, including autoimmune myocarditis, vasculitis including Churg-Strauss syndrome, giant cells arteritis, Kawasaki's disease, polyarteritis nodosa, Takayasu's arteritis and Wegener's granulomatosis; endocrine autoimmune diseases, including Addison's disease, autoimmune hypoparathyroidism, autoimmune hypophysitis, autoimmune oophoritis, autoimmune orchitis, Grave's Disease, Hashimoto's thyroiditis, polyglandular autoimmune syndrome type 1 (PAS-1) polyglandular autoimmune syndrome type 2 (PAS-2), and polyglandular autoimmune syndrome type 3 (PAS-3); autoimmune gastroenteric diseases including autoimmune hepatitis, primary biliary cirrhosis, inflammatory bowel disease, celiac disease, Crohn's disease; autoimmune nervous diseases, including multiple sclerosis, myasthenia gravis, Guillan-Barre syndrome and chronic inflammatory demyelinating neuropathy; and systemic autoimmune diseases including systemic lupus erythematosus, antiphospholid syndrome, autoimmune lymphoproliferative disease, autoimmune polyendocrinopathy, Bechet's disease, Goodpasture's disease, arthritis, including rheumatoid arthritis, osteoarthritis and septic arthritis, sarcoidosis, scleroderma and Sjogren's syndrome, among others.

The term “systemic lupus erythematosus”, “SLE” or “lupus” is used to describe a chronic potentially debilitating or fatal autoimmune disease in which the immune system attacks the body's cells and tissue, resulting in inflammation and tissue damage. LSE refers to several forms of an immunologic disease that affects the joints, skin, muscles, face and mouth, kidneys, central nervous system and other parts of the body. SLE is a chronic and inflammatory disease that can potentially be fatal. SLE can either be classified as an autoimmune or a rheumatic disease. Changes in symptoms are called flares and remissions. Flares are periods when SLE becomes more active with increased symptoms, and remissions are periods when few or no symptoms of lupus are present. In the United States alone, an estimated 270,000 to 1.5 million or more people have SLE, with an estimated 5 million worldwide, having the disease. It is more common than cystic fibrosis or cerebral palsy.

The specific cause of SLE is unknown. It is considered to be a multifactoral condition with both genetic and environmental factors involved. In a multifactoral condition, a combination of genes from both parents, in addition to unknown environmental factors, produce the trait, condition, or disease. It is known that a group of genes on chromosome 6 that code for the human leukocyte antigens play a major role in a person's susceptibility or resistance to the disease. The specific HLA antigens associated with SLE are DR2 and DR3. When the immune system does not function properly, it loses its ability to distinguish between its own body cells and foreign cells. Antinuclear antibodies are autoantibodies (antibodies that fight the body's own cells) that are produced in people with SLE. They often appear in the blood of a patient with SLE.

Studies suggest that some people may inherit the tendency to get SLE, and new research suggests that new cases of SLE appear to be more common in families in which one member already has the disease. However, there is no evidence that supports that SLE is directly passed from parent to child. Females in their childbearing years (18-45) are eight to ten times more likely to acquire SLE than men, and children and the elderly can also acquire the disease.

SLE is unpredictable, and no two people have exactly the same manifestations of the disease. There are 11 criteria that help doctors tell the difference between people who have SLE and people who have other connective tissue diseases. If a person displays 4 or more of the following 11 criteria, the person fulfills the requirement for the diagnosis of SLE.
1. Malar rash—a butterfly shaped rash over the cheeks and across the bridge of the nose;
2. Discoid rash—scaly, disk-shaped sores on the face, neck, and chest;
3. Serositis—inflammation of the lining around the heart, lungs, abdomen, causing pain and shortness of breath;
4. Photosensitivity-skin rash as an unusual reaction to sunlight;
5. Sores or ulcers on the tongue, mouth, or in the nose;
6. Arthritis;
7. Kidney disorder—persistent protein or cellular casts in the urine;
8. Central nervous system problems including seizures and psychosis;
9. Blood problems such as low white blood cell count, low lymphocyte count, low platelet count, or hemolytic anemia;
10. Immune system problems (immune dysfunction/dysregulation)—presence of abnormal autoantibodies to double stranded DNA, Sm antigen or phospholipid in the blood; and
11. Presence of abnormal antinuclear antibodies in the blood.

Other symptoms/manifestations of SLE include inflammatory lung problems lymphadenopathy, fever, nausea, vomiting, diarrhea, swollen glands, lack of appetite, sensitivity to cold (Raynaud's phenomenon), weight loss, and hair loss.

Notwithstanding the numerous disease states, conditions and/or manifestations associated with SLE, it is difficult to diagnose because there is no single set of signs and symptoms to determine if a person has the disease. There is no single test that can diagnose. SLE. Some tests used to diagnose SLE include urinalysis to detect kidney problems, tests to measure the amount of complement proteins in the blood, complete blood cell counts to detect hematological disorders, and an ANA test to detect antinuclear antibodies in the blood. Additionally, X-rays may be ordered to check for lung and heart problems.

The term “treatment” or “treating” is used to describe an approach for obtaining beneficial or desired results including and preferably clinical results. For purposes of this invention, beneficial or desired clinical results include, but are not limited to, one or more of the following: alleviation of one or more symptoms, diminishment of the extent of disease, stabilized (i.e., not worsening) state of disease, preventing or reducing the likelihood of the spread of disease, reducing the likelihood of occurrence or recurrence of disease, decreasing, delaying or reducing the likelihood of the occurrence of “flares” or “attacks”, for example, in the case of SLE, amelioration of the disease state, remission (whether partial or total), reduction of incidence of disease and/or symptoms, stabilizing (i.e., not worsening) of immune or renal function or improvement of immune or renal function. “Flares” refer to an increase in activity, generally inflammatory activity in a particular tissue. The “treatment” of autoimmune or immune inflammatory diseases, including SLE, may be administered when no symptoms of autoimmune disease or SLE are present, and such treatment (as the definition of “treatment” indicates) reduces the incidence or likelihood of flares or other symptoms. Also encompassed by “treatment” is a reduction of pathological consequences of any aspect of an autoimmune disease, SLE or any associated disease states or conditions, including immune inflammatory diseases, including skin rashes (malar and discoid), arthritis, serositis (inflammation of the lining around the heart, lungs, abdomen), sores (mouth, nose and tongue), immune dysfunction/dysregulation, central nervous system problems (including psychosis, seizures and strokes), blood problems (including low white blood cell count, low platelet count, or anemia), the presence of antinuclear antibodies in the blood and kidney disease/dysfunction (especially SLE-related nephritis). Symptoms of autoimmune disease vary widely depending on the type of disease. A group of very nonspecific autoimmune symptoms often accompany autoimmune diseases especially of the collagen vascular type and include fatigue, dizziness, malaise and fever, including low-grade temperature elevations. More specific symptoms of autoimmune disease include the destruction of an organ or tissue resulting in decreased functioning of that organ or tissue (for example, the islet cells of the pancreas are destroyed in diabetes) and the increase or decrease in the size of an organ or tissue, for example, thyroid enlargement in Grave's disease. Symptoms generally vary with the specific disorder and the organ or tissue affected. In at least one treatment aspect of the present invention, the reduction, control or elimination of symptoms of an autoimmune disease in a patient is an important feature.

“Flares” are used herein to refer to flares (i.e. acute clinical events) which occur in patients with SLE or other autoimmune diseases. SLE flares may be in various major organs, including but not limited to, kidney, brain, lung, heart, liver, connective tissues and skin. Flares can include activity in all tissues that may be affected by SLE. Remission is a term used to refer to periods of little or no lupus or other autoimmune symptoms.

“Reducing incidence” of renal flares in an individual with SLE means any of reducing severity (which can include reducing need for and/or amount of (e.g., exposure to) other drugs generally used for this conditions, including, for example, high dose corticosteroid and/or cyclophosphamide), duration, and/or frequency (including, for example, delaying or increasing time to renal flare as compared to not receiving treatment) of renal flare(s) in an individual. As is understood by those skilled in the art, individuals may vary in terms of their response to treatment, and, as such, for example, a “method of reducing incidence of renal flares in an individual” reflects administering the conjugate(s) described herein based on a reasonable expectation that such administration may likely cause such a reduction in incidence in that particular individual.

The present invention relates to the use of a GSK3 inhibitor as otherwise described herein in effective amounts for inducing immunological tolerance in a patient in need thereof. In alternative aspects of the invention, GSK3 inhibitors lead to activation of a pathway E-cadherin/β-catenin of dendritic cell maturation which leads to a dendritic phenotype which attenuates, rather than, induces, immune responses. The immune responses and mature dendritic cells produced by the method of the present invention attenuate the immune response in individuals, thus leading to effective therapies for a number of autoimmune diseases and/or diseases of immune dysfunction/dysregulation, including systemic lupus erythematosus (SLE), autoimmune diabetes (type I diabetes mellitus), asthma, rheumatoid arthritis, inflammatory bowel disease, among numerous others.

In the present invention, a GSK3 inhibitor is administered in an effective amount to a patient exhibiting immune intolerance/immune dysfunction in order to induce immune tolerance in the patient. In at least one aspect of the invention, the method of the present invention results in the activation of the E-cadherin/β-catenin pathway in immature dendritic cells and the production of mature dendritic cells which exhibit a T cell response associated with the induction or maintenance of T cell tolerance in said patient. The resulting mature dendritic cells which are produced from the use of GSK inhibitors according to the present invention, are characterized by many features of dendritic cells which are matured by microbial stimuli, such as increase in major histocompatibility complex (MHC) type II products, costimulatory molecules (e.g. CD80, CD86) and the upregulation of chemokine receptors required for dendritic cell migration.

The present invention also relates to the treatment of autoimmune disease in a patient or subject, in particular a human subject. The present invention relates to administering a GSK3 inhibitor as otherwise disclosed herein in an effective amount, optionally in combination with a pharmaceutically acceptable carrier, additive or excipient to a patient or subject in need of therapeutic treatment of an autoimmune disease. Autoimmune disease which may treated using the present invention include, for example, systemic lupus erythematosis (SLE), diabetes mellitus (type I), asthma, Grave's disease, arthritis, including rheumatoid arthritis and osteoarthritis, pernicious anemia, and multiple sclerosis, among numerous others. Other autoimmune diseases may be treated using the method of the present invention including autoimmune blood diseases, including pernicious anemia, autoimmune hemolytic anemia, aplastic anemia, idiopathic thrombocytopenic purpura, ankylosing spondylitis; autoimmune diseases of the musculature including polymyositis and dermatomyositis, autoimmune diseases of the ear including autoimmune hearing loss and Meniere's syndrome, autoimmune eye diseases, including Mooren's disease, Reiter's syndrome and Vogt-Koyanagi-Harada disease, autoimmune diseases of the kidney including glomerulonephritis and IgA nephropathy; diabetes mellitus (type I); autoimmune skin diseases including pemphigus (autoimmune bullous diseases), such as pemphigus vulgaris, pemphigus foliaceus, pemphigus erythematosus, bullous pemphigoid, vitiligo, epidermolysis bullosa acquisita, and alopecia areata; cardiovascular autoimmune diseases, including autoimmune myocarditis, vasculitis including Churg-Strauss syndrome, giant cells arteritis, Kawasaki's disease, polyarteritis nodosa, Takayasu's arteritis and Wegener's granulomatosis; endocrine autoimmune diseases, including Addison's disease, autoimmune hypoparathyroidism, autoimmune hypophysitis, autoimmune oophoritis, autoimmune orchitis, Grave's Disease, Hashimoto's thyroiditis, polyglandular autoimmune syndrome type 1 (PAS-1) polyglandular autoimmune syndrome type 2 (PAS-2), and polyglandular autoimmune syndrome type 3 (PAS-3); autoimmune gastroenteric diseases including autoimmune hepatitis, primary biliary cirrhosis, inflammatory bowel disease, celiac disease, Crohn's disease; autoimmune nervous diseases, including multiple sclerosis, myasthenia gravis, Guillan-Barre syndrome and chronic inflammatory demyelinating neuropathy; and systemic autoimmune diseases including systemic lupus erythematosus, antiphospholid syndrome, autoimmune lymphoproliferative disease, autoimmune polyendocrinopathy, Bechet's disease, Goodpasture's disease, arthritis, including rheumatoid arthritis, osteoarthritis and septic arthritis, sarcoidosis, scleroderma and Sjogren's syndrome, among others.

The term “coadministration” or “combination therapy” is used to describe a therapy in which at least two active compounds in effective amounts are used to treat a viral infection at the same time. Although the term coadministration preferably includes the administration of two active compounds to the patient at the same time, it is not necessary that the compounds be administered to the patient at the same time, although effective amounts of the individual compounds will be present in the patient at the same time. The term coadministration shall generally refer to at least one GSK3 inhibitor in combination with at least one additional GSK3 inhibitor, or alternatively, at least one additional compound which is used to treat an autoimmune disease. For example, for the treatment of autoimmune diseases, especially SLE, these agents may include, for example, non-steroidal anti-inflammatory drugs (NSAIDs) including traditional NSAIDs, COX-2 inhibitors and salicylates (such as aspirin), anti-malarials such as hydroxychloroquine, quinacrine, corticosteroids such as prednisone (Deltasone), betamethasone (Celestone), methylprednisolone acetate (Medrol, Depo-Medrol), hydrocortisone Cortef, Hydrocortone) and dexamethasone (Decadron, Hexadrol), among others and immunosuppressants such as methotrexate (Rhematrex), cyclophosphamide (cytoxan), Azathioprine (Imuran) and mycophenolate mofetil (MM, also Cellsept). In the case of diabetes, one or more compound according to the present invention is generally coadministered with insulin.

In method aspects according to the present invention, a GSK3 inhibitor in combination with a pharmaceutically acceptable carrier additive or excipient is administered alone or in combination with another agent to a patient or subject in an effective amount to induce immune tolerance in said subject or patient. In the present invention, the inhibition of GSK3 leads to activation of a pathway (E-cadherin/β-catenin pathway in immature dendritic cells) of dendritic cell maturation which leads to a mature dendritic phenotype (in mature dendritic cells) which attenuates or induces immune tolerance, rather than enhancing immune responses. The immune responses and mature dendritic cells produced by the method of the present invention attenuate the immune response in individuals, thus leading to effective therapies for a number of autoimmune diseases and/or diseases of immune dysfunction/dysregulation, including systemic lupus erythematosus (SLE), autoimmune diabetes (type I diabetes mellitus), asthma, rheumatoid arthritis, inflammatory bowel disease, among numerous others as otherwise described herein.

Thus in another aspect, the present invention relates to the use of a GSK3 inhibitor, in particular, an inhibitor of GSK-3α, GSK-3β and GSK-3β2, especially GSK-3β, for the treatment of an autoimmune disease comprising administering to a patient or subject the GSK3 inhibitor, alone or in combination with another active agent in combination with a pharmaceutically acceptable carrier, additive or excipient, wherein the autoimmune disease is systemic lupus erythematosis (SLE), diabetes mellitus (type I), asthma, autoimmune blood diseases, including pernicious anemia, autoimmune hemolytic anemia, aplastic anemia, idiopathic thrombocytopenic purpura, ankylosing spondylitis; autoimmune diseases of the musculature including polymyositis and dermatomyositis, autoimmune diseases of the ear including autoimmune hearing loss and Meniere's syndrome, autoimmune eye diseases, including Mooren's disease, Reiter's syndrome and Vogt-Koyanagi-Harada disease, autoimmune diseases of the kidney including glomerulonephritis and IgA nephropathy; diabetes mellitus (type I); autoimmune skin diseases including pemphigus (autoimmune bullous diseases), such as pemphigus vulgaris, pemphigus foliaceus, pemphigus erythematosus, bullous pemphigoid, vitiligo, epidermolysis bullosa acquisita, and alopecia areata; cardiovascular autoimmune diseases, including autoimmune myocarditis, vasculitis including Churg-Strauss syndrome, giant cells arteritis, Kawasaki's disease, polyarteritis nodosa, Takayasu's arteritis and Wegener's granulomatosis; endocrine autoimmune diseases, including Addison's disease, autoimmune hypoparathyroidism, autoimmune hypophysitis, autoimmune oophoritis, autoimmune orchitis, Grave's Disease, Hashimoto's thyroiditis, polyglandular autoimmune syndrome type 1 (PAS-1) polyglandular autoimmune syndrome type 2 PAS-2), and polyglandular autoimmune syndrome type 3 (PAS-3); autoimmune gastroenteric diseases including autoimmune hepatitis, primary biliary cirrhosis, inflammatory bowel disease, celiac disease, Crohn's disease; autoimmune nervous diseases, including multiple sclerosis, myasthenia gravis, Guillan-Barre syndrome and chronic inflammatory demyelinating neuropathy; and systemic autoimmune diseases including systemic lupus erythematosus, antiphospholid syndrome, autoimmune lymphoproliferative disease, autoimmune polyendocrinopathy, Bechet's disease, Goodpasture's disease, arthritis, including rheumatoid arthritis, osteoarthritis and septic arthritis, sarcoidosis, scleroderma and Sjogren's syndrome, among others.

The pharmaceutical composition used in the present invention may be in a form chosen from sterile isotonic aqueous solutions, pills, drops, pastes, cream, spray (especially including aerosols from pulmonary administration in the case of asthma), capsules, tablets, sugar coating tablets, granules, suppositories, liquid, lotion, suspension, emulsion, ointment, gel, and the like. Administration route may be chosen from subcutaneous, intravenous, intestinal/rectal, parenteral (including intravenous), oral, pulmonary (especially for treatment of lung conditions, including asthma), buccal, nasal, intramuscular, transcutaneous, transdermal, intranasal, intraperitoneal, and topical (especially for certain autoimmune skin rashes and skin conditions).

The subject or patient may be chosen from, for example, a human, a mammal such as domesticated animal, or other animal. The subject may have one or more of the disease states, conditions or symptoms associated with one or more autoimmune disease as otherwise described herein.

The compounds according to the present invention may be administered in an effective amount to treat or reduce the likelihood of an autoimmune disease, any one or more of the disease states or conditions associated with an autoimmune disease. In the case of SLE these include, for example, serositis, malar rash (rash over the cheeks and bridge of the nose), discoid rash (scaly, disk-shaped sores on the c, neck and chest), sores or ulcers (on the tongue, in the mouth or nose), arthritis, hemolytic anemia, low lymphocytic count, low platelet count, the presence of antinuclear bodies in the blood, skin lesions, CNS effects (including loss of memory, seizures, strokes and psychosis), lung symptoms/effects including inflammation (pleuritis), chronic pneumonitis, chronic diffuse interstitial lung disease and scarring of the lungs, hair loss, Raynaud's syndrome, lupus nephritis and sensitivity to light, fatigue, fever, nausea, vomiting, diarrhea, swollen glands, lack of appetite, sensitivity to cold (Raynaud's phenomenon) and weight loss. One of ordinary skill in the art would be readily able to determine an effective amount of an agent by taking into consideration several variables including, but not limited to, the animal subject, age, sex, weight, site of the disease state or condition in the patient, previous medical history, other medications, etc.

For example, the dose of a compound for a human patient is that which is an effective amount and may range from as little as 10 (preferably at least about 100) μg to at least about 500 mg or more, which may be administered in a manner consistent with the delivery of the drug and the disease state or condition to be treated. In the case of oral administration, active is generally administered from one to four times or more daily. In the case of asthma, active may be administered from one to four times daily or in the event of an asthma attack Transdermal patches or other topical administration may administer drugs continuously, one or more times a day or less frequently than daily, depending upon the absorptivity of the active and delivery to the patient's skin. Of course, in certain instances where parenteral administration represents a favorable treatment option, intramuscular administration or slow IV drip may be used to administer active. The amount of an active compound which is administered to a human patient may range from about 0.05 mg/kg to about 20 mg/kg, depending on the compound used.

The dose of a GSK3 inhibitor according to the present invention may be administered prior to the onset of an autoimmune flare or attack, during a flare or attack or during remission prior to an expected flare or attack. For example, the dose may be administered for the purpose of treating and/or reducing the likelihood of any one or more of these disease states or conditions occurs or manifests. In the case of SLE this will include serositis, malar rash (rash over the cheeks and bridge of the nose), discoid rash (scaly, disk-shaped sores on the face, neck and chest), sores or ulcers (on the tongue, in the mouth or nose), arthritis, hemolytic anemia, low lymphocytic count, low platelet count, the presence of antinuclear bodies in the blood, skin lesions, CNS effects (including loss of memory, seizures, strokes and psychosis), lung effects including chronic pneumonitis and scarring of the lung, hair loss, Raynaud's syndrome, lupus nephritis, sensitivity to light, fatigue, fever, nausea, vomiting, diarrhea, swollen glands, lack of appetite, sensitivity to cold (Raynaud's phenomenon), weight loss, and hair loss. The dose may be administered prior to diagnosis, but in anticipation of an autoimmune disease flare or attack. The dose may also be administered during flares to reduce the severity of same.

### Disruption of E-Cadherin-Mediated Contacts Induce DC Maturation

It has long been observed in primary bone marrow-derived cultures that differentiating DCs form clusters and exhibit spontaneous maturation when the clusters are inadvertently disaggregated (Pierre et al., 1997). Since several components of tight and adherence junctions have been observed in murine DCs (at the mRNA level) (Rescigno et al., 2001), it was conceivable that loss of E-cadherin contacts induced the maturation of bone marrow-derived DCs (BMDCs). Indeed, flow cytometry revealed that CD 11c⁺ BMDCs exhibited high amounts of surface E-cadherin after 5-6 days in culture, which remained high even after stimulation with the TLR4 agonist LPS (Suppl. FIG. 2A). Importantly, E-cadherin was responsible for maintaining the cell clusters in these cultures, as they could be dissociated by addition of an inhibitory E-cadherin mAb, as shown previously for LCs (Suppl. FIG. 2B). Physical disruption of the BMDC clusters (accomplished by passing them over magnetic columns), however, triggered all of the morphological features of DC maturation (Mellman and Steinman, 2001). These include the redistribution of MHC class II molecules from lysosomes to the cell surface (FIG. 2A), the down regulation of macropinocytosis (not shown), the upregulation of costimulatory molecules and ability to present peptide to antigen-specific T cells (Suppl. FIG. 2C). Maturation was due at least in part to the loss of E-cadherin contacts, since it could be prevented by adding the E-cadherin blocking antibody either before or during cluster disruption (CD) (FIG. 2B). Maturation was not blocked using non-specific mAb or isotype-matched mAb against the DC integrin CD11b (FIG. 2B), nor did anti-E-cadherin inhibit maturation induced by LPS (see below). Analogous results were obtained using human CD34⁺-derived LCs (not shown).

### Disruption of E-Cadherin-Mediated Adhesion Activates a β-Catenin-TCF/LEF Signaling Pathway Independent of TLR Signaling

DC maturation is exceedingly sensitive to minute amounts of contaminating LPS. To determine if contaminating LPS contributed to the E-cadherin-induced maturation, we compared the signaling events induced by CD to those induced by LPS stimulation of TLR4. TLR signaling is well known to be associated with the activation of NF-κB and p38 MAPK (Barton and Medzhitov, 2003; Takeda and Akira, 2004). As expected, LPS induced a strong activation of both signaling pathways, as revealed by the phosphorylation of IκBα and p38 MAPK (FIG. 3A). In contrast, neither p38 MAPK nor IκBα was detectably phosphorylated following CD (FIG. 3A). In addition, TLR4^{-/-} DCs, which do not respond to LPS, exhibited robust maturation following CD (Suppl. FIG. 3A), further demonstrating that CD signals maturation independently by a mechanism that is distinct from that due to TLR agonists.

The fact that alterations in E-cadherin interactions induced DC maturation raised the possibility that maturation involved the activation of β-catenin. We therefore used a monoclonal antibody that specifically recognizes a non-phosphorylated form of β-catenin induced after canonical Wnt signaling (van Noort et al., 2002). As shown in FIG. 2B. CD caused the accumulation of non-phosphorylated β-catenin relative to control or LPS-treated DCs. The active β-catenin was apparently cytosolic as it was not co-precipitated with anti-E-cadherin (FIG. 3B).

We next asked whether CD could drive activation of the β-catenin-dependent transcriptional activators TCF/LEF. This was investigated using two different reporter systems. First, DCs were transduced with retroviruses encoding EGFP under the control of an optimal TCF promoter (TOP-EGFP) or with a construct containing inactive mutant promoter (FOP-EGFP) (Korinek et al., 1997). EGFP production was monitored by flow cytometry after CD. Untreated control DCs expressed similar amounts of EGFP (FIG. 3C, left). 48 hr after CD treatment, however, there was a significant increase in EGFP expression in the TOP-EGFP transfected DCs compared to the FOP-EGFP expressing DCs (FIG. 3C, right), indicating that cluster disruption activated TCF/LEF-dependent transcription. Although the signal obtained using the EGFP reporter was less than that obtained after Wnt activation using a luciferase assay with the same reporter system, unlike luciferase, EGFP signals are not amplified. Indeed, a similarly modest EGFP increase was observed in MDCK cells expressing the same constructs following Wnt activation by Lithium treatment (which inhibits GSK30 (Suppl. FIG. 3B). A >10 fold increase was generated using the luciferase assay in MDCK cells under the same conditions (Lyons et al., 2004). In our experiments, it was necessary to use an EGFP reporter to identify the small fraction (<10%) of productively infected DCs by flow cytometry.

To overcome the quantitative limitation of retrovirus approach, we next took advantage of transgenic mice that uniformly express the TOPGAL reporter (DasGupta and Fuchs, 1999). CD as well as LPS treatment resulted in strong maturation with DCs prepared from transgenic mice (FIG. 3D, left). However, only cluster disruption produced a significant increase in β-galactosidase activity, indicative of TCF/LEF activation (FIG. 3D, right).

Together, the reporter assays provide direct evidence that disruption of E-cadherin-mediated adhesion activates the β-catenin-TCF/LEF signaling pathway in DCs.

### Activation of β-Catenin Signaling Plays a Role in DC Maturation

To determine if activation of β-catenin signaling pathway might actually contribute to DC maturation, we first used a pharmacologic approach. SB216763 is a selective inhibitor of GSK3β (Coghlan et al., 2000), the kinase whose phosphorylation of β-catenin marks it for degradation by the proteasome. As expected, treatment of immature cells with SB216763 resulted in a dose-dependent accumulation of β-catenin in the cytosol in both murine (FIG. 4A) and human DCs (not shown), as assayed by cell fractionation and Western blot.

SB216763 not only stabilized β-catenin, but also was a potent inducer of DC maturation. When the GSK3β inhibitor was added to immature DCs, the mature population (defined as the percentage of cells expressing high CD86) increased in a dose-dependent manner (FIG. 4B). Indeed, at 10 μM, SB216763 was nearly as effective as LPS at triggering DC maturation, and slightly more effective than CD. Similar results were obtained for human CD34⁺-derived DCs (not shown). By immunofluorescence, it was clear that DCs treated with the inhibitor assumed the classical mature DC phenotype, with MHC class II molecules redistributing from lysosomes to the plasma membrane (not shown; cf FIG. 2A).

Although these results strongly suggested that activation of β-catenin by either CD or inhibition of GSK3β led to DC maturation, both treatments may have relevant downstream targets other than β-catenin. Therefore, we asked if a selective increase in β-catenin could induce DC maturation. A recombinant retrovirus was used to express β-catenin-GFP. Expression of β-catenin-GFP, but not of GFP alone, resulted in a significant increase in the fraction of CD86-high mature DCs (FIG. 4C). Similar results were obtained using a virus encoding a stabilized mutant β-catenin-GFP (not shown). Although the extent of CD86 upregulation was not as great as found for CD or SB216763 treatment, the GFP tag may have interfered with β-catenin, or expression may have been too low for optimal maturation. Alternatively, β-catenin may indeed work synergistically with other components that may be targets of GSK3β or E-cadherin activation.

Finally, if β-catenin can induce DC maturation, it would be expected to enter the nucleus. Indeed when the intracellular distribution of β-catenin of β-catenin-GFP transfected DCs was determined by confocal microscopy following CD, it was detected in the nucleus (FIG. 4C inset, arrow). Taken together, these results strongly suggest that activation of the β-catenin signaling pathway, by CD, inhibition of GSK3□, or expression of exogenous β-catenin can at least in part induce a mature DC phenotype.

### DCs Matured by Cluster Disruption Exhibit a Distinct Transcriptional Profile

We next performed a genome-wide microarray analysis to study the expression profiles of DCs matured by CD as opposed to a conventional TLR agonist (E. coli bacteria). RNA was isolated from human CD34⁺ DCs at various times after stimulation and used to probe Affymetrix U95Av2 chips. >700 genes were found differentially regulated upon maturation by either CD or bacterial stimulation (FIG. 5A and Suppl. FIG. 4). Cluster analysis revealed that following an early phase (1-3 hr) of similarity, expression profiles exhibited by the two sets of DCs diverged significantly at later time points (>6 hr) (FIG. 5A). A large number of transcripts were markedly upregulated (red) in the bacteria-stimulated set that remained relatively unchanged or actually decreased (blue) in the cluster-disrupted set. There were some transcripts upregulated in cluster-disrupted cells, however, with at least some of these increases prevented by including anti-E-cadherin mAb under conditions that blocked maturation. Clearly, the transcriptional events induced by alteration of E-cadherin-mediated adhesion were quite distinct from those induced by TLR activation.

We next asked if any targets of β-catenin-induced transcription were upregulated in DCs stimulated by CD. Guided by the gene set compiled for various cell types by R. Nusse and colleagues (see Stanford-edu/˜rnusse/pathways/targets.html on the world wide web), we identified increases in at least 10 β-catenin-TCF/LEF targets, including: Ephb2, TCF1, CD44, FZD7 (Frizzled homolog 7; a component of the Wnt pathway), VEGF, cyclin D2, Ephb3, and Id2 (FIG. 5B). Each of these inductions occurred after a lag of 1-3 hr and each was partially inhibited by anti-E-cadherin mAb (FIG. 5B), suggesting that CD activated at least some elements of the β-catenin signaling pathway.

We then quantified the extent to which selected transcripts were upregulated by CD vs. TLR stimulation (FIG. 5C). Several were enhanced by both stimuli including FZD7 as well as the chemokines TARC and MCP-1. The chemokines IL-8 (CXCL8) and CCL22 as well as the adhesion protein CD44 were also enhanced by both stimuli, albeit to a greater extent by bacteria. There were also some differences. For instance, a cluster of genes including chemokine receptor CX₃CR1 was strongly downregulated by TLR signaling but not by CD (FIG. 5C, lower left). Few if any genes were upregulated to a greater extent by CD than by TLR stimulation (with TARC and Wnt10b [not shown] as potential exceptions). Most striking, however, was the fact that genes encoding inflammatory cytokines, such as IL6, were dramatically upregulated by TLR stimulation, but not at all by CD (FIG. 5C, lower right).

### DCs Matured by Cluster Disruption Neither Produce Nor Secrete Inflammatory Cytokines

Next we directly measured cytokine released into the media. Treatment of human DCs with bacteria greatly enhanced release of the inflammatory cytokines IL-1α, IL-6, TNF-α, and IL12 p40 24-48 hr after stimulation (FIG. 5D). In contrast, DCs matured by CD failed to secrete any of these cytokines above background levels. Consistent with the array results, secretion of several chemokines including CXCL8, MCP-1 and MIP-1α were significantly enhanced after CD, albeit in amounts far lower than DCs matured by bacteria (FIG. 4D and data not shown). Thus, activation via E-cadherin upregulated the chemokine pathway without inducing inflammatory cytokine production in human CD34⁺-derived DCs.

We next asked if E-cadherin-mediated maturation similarly failed to induce inflammatory cytokine production by murine BMDCs. Using real time RT-PCR, we observed that BMDCs matured by CD did not induce significant increases in transcription of genes encoding TNF-α, IL-6, IL-1α, or IL-12p40. In contrast, LPS treatment resulted in dramatic (if sometimes transient) increases in each of these inflammatory markers (FIG. 6A).

Interestingly, CD treatment of BMDCs did lead to upregulation of CCR7, both at the mRNA level (by RT-PCR) and on the plasma membrane (by FACS) (FIG. 6B and data not shown). CCR7 is a chemokine receptor important for the migration of DCs from the periphery to T cell areas of lymph nodes (Randolph et al., 2005), so that in principle, activation of the E-cadherin pathway in vivo would result in cells capable of migrating to secondary lymphoid organs. Taken together, these results suggested that the loss of E-cadherin-mediated adhesion might provide a spatial cue for the generation of mature, migratory DCs but without the ability to induce T cell immunity.

To determine if the two maturation signals synergize or compete with each other, we next examined the cytokine profiles of DCs matured by LPS alone or by both CD and LPS. In general, addition of LPS at the time of CD yielded a phenotype more similar to LPS alone than to CD alone (TNF-α, IL-6, IL-1α, and IL12 p40; FIG. 6C). This finding suggested that the TLR signal was dominant to the E-cadherin-induced signal, at least when presented simultaneously. A rather different set of results was obtained if LPS was added to DCs that had been matured by CD for over 12 hr beforehand. LPS could no longer induce the transcription of IL-10 mRNA, and only partly induced IL-6 and TNF-α transcription. Transcription of IL-12p40 and IL-1 α, on the other hand, was more effectively induced (FIG. 6C and not shown). Thus, although LPS could enhance cytokine secretion if provided during the CD step, prior E-cadherin activation either prevented or enhanced (in the case of IL-12) the LPS effects.

This prompted us to investigate whether different maturation signals regulated the ability to activate naïve T cells. Fixed DCs that had been pulsed with OVA protein and matured by different treatments were tested for their ability to activate OVA-specific OTII CD4 and OTI CD8 T cells in vitro. DCs matured by CD elicited both CD4 and CD8 T cell responses, while LPS-matured DCs were able to stimulate only CD4 T cells (Suppl. FIG. 5). Although this observation was suggested previously (Delamarre et al., 2003), the current data allow a direct, quantitative comparison of both maturation signals. Indeed, sequential treatment by CD followed by LPS caused a rather substantial synergistic increase in cross-presentation to OTI cells. These data emphasize that cluster disruption matures DCs through a mechanism distinct to that due to TLR signaling. For both CD8 and CD4 responses, the addition of LPS to CD-matured DCs greatly enhanced the extent of T cell activation (Suppl FIG. 5), consistent with the synergy observed in cytokine production. Thus, E-cadherin-induced maturation program could modulate subsequent LPS stimulation to enhance T cell response.

### DCs Matured by E-Cadherin and TLR Activation Elicit Distinct T Cell Responses In Vivo

Since DCs matured by CD alone could efficiently present antigen but did not produce inflammatory cytokines, we predicted that they would not be immunogenic in vivo. To test this possibility, we compared the ability of DCs matured by CD or CD followed by LPS to elicit T cell responses in mice. Both types of mature DCs were incubated in vitro with OVA-derived peptides and then injected into C57B/6 mice. Consistent with our in vitro results, immunization with either population of mature DCs led to the proliferation of adoptively transferred CD4 and CD8 T cells transgenic for OVA-specific T cell receptors (not shown). Thus, the injected DCs could encounter and stimulate naïve, but antigen-specific T cells in vivo.

We next asked if the DCs could actually stimulate immunity: could they prime naïve CD4 T cells to become IFN-γ-producing effectors in vivo? DCs were matured by CD alone or by CD and LPS, loaded with OVA peptide 323-339, and injected three times during a one week period (see Methods). Three days after the last injection, splenocytes were isolated and restimulated in vitro with the OVA peptide. While sequentially matured DCs led to a dramatic increase in the production of IFN-γ during this recall response, DCs matured by CD alone failed to prime CD4 T cells to produce IFN-γ but did generate high levels of IL10 (FIG. 7A), a cytokine profile consistent with the presence of type I regulatory T cells (Tr1) (O'Garra and Vieira, 2004). Furthermore, while both DCs expanded the overall population of activated CD4⁺CD25⁺ T cells to similar extents (not shown), only immunization with DCs that had been matured sequentially by CD and LPS generated IFN-γ-producing CD4 T cells (FIG. 7B, middle). In stark contrast, DCs matured by CD induced only IL10-producing T cells (FIG. 7B, top). Additionally, DCs matured by CD failed to generate a significant antibody response as compared to DCs matured by both CD and LPS (Jiang A and Mellman I, unpublished observations). Thus, DCs matured by CD alone were not immunogenic and instead induced the production of IL10-producing, putative regulatory T cells.

### Discussion

One of the most intriguing specializations of DCs is the process of maturation (Trombetta and Mellman, 2005). The term was originally used to describe the acquisition of antigen-presenting activity by murine epidermal LCs after isolation from skin (Romani et al., 2003). More recently, maturation has come to describe a panoply of functional and morphological transformations triggered by TLR ligands, microbial products, inflammatory cytokines, or T cell surface proteins (eg CD40 ligand) (Mellman and Steinman, 2001). We can now add another mediator of DC maturation to this list, the activation following alterations in E-cadherin-mediated cell adhesion. Although we have not completely defined the biochemical features or physiological significance of this new pathway, we have established its functional consequences in vitro and in vivo and found them to be strikingly different from virtually all other pathways of DC maturation.

E-cadherin-induced maturation is unique on several accounts. First, it is not triggered by components associated with the inflammatory response or microbial invasion. The E-cadherin/□-catenin pathway is best known for its role in the function of epithelial tissues and in organogenesis. In these examples, the sequestration of β-catenin by cadherins helps to regulate Wnt signaling and thereby cell proliferation and morphogenesis (Nelson and Nusse, 2004). In addition, induced alterations in homotypic cadherin interactions may play a direct role in triggering β-catenin signaling in endothelial cells during angiogenesis (Dejana, 2004). Although we cannot conclude that alterations in E-cadherin-mediated adhesion act alone (eg in the absence of a Wnt signal) or functions exclusively by activating the β-catenin pathway, our data do strongly suggest that β-catenin signaling is at least partly involved.

Second, unlike other signals studied thus far, induction of maturation by E-cadherin can occur under steady state conditions. LCs and possibly all other DCs that reside in peripheral tissues interact with neighboring cells by adhesion mediated via E-cadherin or related members of the cadherin family. Disruption of these interactions, which would occur concomitant with tissue emigration, would thus lead to the activation of DC maturation even in the complete absence of infection or inflammation. That there is continuous traffic of DCs from tissues is clear, but it is unclear what triggers the loss of adhesion. As monomers, cadherin interactions are relatively low affinity, with adhesive strength reflecting the contribution of multiple cadherins at contact sites. Cadherin-mediated adhesions are also dependent on links to the actin cytoskeleton via β-catenin, which binds to the cadherin cytoplasmic domain via β-catenin. Thus, any physical disruption (analogous to that performed in vitro) that dissociates even a few E-cadherin interactions may be sufficient to reduce the strength of a given contact to induce maturation and migration. The mild mechanical trauma that occurs continuously in the skin might serve this purpose, and has already been associated with LC traffic to lymph nodes (Jakob et al., 2001). Similarly, in motile organs such as the gut, E-cadherin has been implicated in the anchoring of DCs in the mucosa (Rescigno et al., 2001); mild trauma may contribute as well. Alternatively, the steady state production of local agonists (including Wnt) may stochastically signal the dissociation of β-catenin following E-cadherin phosphorylation, weakening adhesive contacts (Dejana, 2004).

Finally, and perhaps most strikingly, the E-cadherin-mediated pathway produces DCs that contains the phenotypic hallmarks of mature DCs but that do not produce inflammatory cytokines. Thus, despite redistributing MHC class II molecules from lysosomal compartments, down regulating endocytosis, upregulating costimulatory molecules and chemokine receptors, and enhancing antigen processing activity, E-cadherin-induced DCs would not be expected to produce a sustained immune response. Such a phenotype might well be associated with the induction of peripheral tolerance in vivo (Pasare and Medzhitov, 2004); tolerance is a function associated with the constitutive traffic of otherwise unstimulated DCs from the periphery to lymph nodes (Steinman et al., 2003). Indeed, while DCs matured upon alteration of E-cadherin-mediated adhesion expanded the activated CD4 T cells as well as DCs matured by LPS, they failed to prime them to become IFN-γ producing effectors, instead, they developed into ILL O-producing cells with characteristics of regulatory T cells (Tr1), consistent with the induction of tolerance (O'Garra and Vieira, 2004). Thus, stimulation of the E-cadherin pathway may represent a signal for generating tolerogenic DCs under steady state conditions.

Signaling and DC function. Although it was established >10 years ago that LCs expressed E-cadherin (Tang et al., 1993) and that E-cadherin interactions may regulate LC maturation (Jakob and Udey, 1998; Riedl et al., 2000a; Riedl et al., 2000b), the underlying mechanism was not explored. E-cadherin in mouse and human DCs forms a complex with β-catenin and p120^{catenin} at the plasma membrane (data not shown). Conceivably, upon cluster disruption, some β-catenin is discharged from the plasma membrane with a fraction translocating to the nucleus. Stabilization of cytosolic β-catenin by inactivating GSK3β (which phosphorylates β-catenin for proteasome destruction) also induced DC maturation as did, albeit to a lesser extent, transfection of immature DCs with β-catenin cDNA Maturation by CD activated β-catenin-TCF/LEF-mediated transcription (mouse DCs) and enhanced the expression of genes associated with β-catenin-TCF/LEF-mediated transcriptional events (human DCs). Although our data clearly established that the alteration of E-cadherin-mediated adhesion alone could lead to activation of β-catenin/TCF pathway, it remains possible that Wnt signaling must also contribute. This might occur following CD, perhaps by the attendant shear force and signaling pathways possibly associated with primary cilia expressed by virtually all cells, events reported to initiate Wnt signaling in other systems (Norvell et al., 2004; Simons et al., 2005).

The ability of the GSK3β inhibitor SB216763 to induce maturation was particularly striking. While it is possible this effect was limited to the dramatic increase in free β-catenin, GSK3β also has other targets. For example, by inhibiting GSK3β-mediated phosphorylation of NF-AT, this transcriptional activator would be more readily translocated into the nucleus, possibly synergyzing with β-catenin (Crabtree and Olson, 2002). Other potential mediators (eg Hedgehog, p53, c-myc) might also be affected (Doble and Woodgett, 2003). However, the ability of transfected β-catenin cDNA to affect at least a partial maturation response strongly suggests that SB216763-induced maturation was at least partly due to a direct effect on β-catenin. SB216763 also induced the formation of the same array of chemokines as did CD (eg IP10, MCP-1, MIP-α, CXCL8) and failed to induce the production of inflammatory cytokines. Interestingly, inhibition of GSK3β in human monocytes was also found to downregulate inflammatory cytokine production while enhancing anti-inflammatory cytokine production by differentially regulating NF-κB and CREB (Martin et al., 2005).

There are only a few signaling pathways in DCs that to date have been found to yield a similar maturation phenotype to CD or GSK3β inhibition. One pathway results from ligation of the orphan plasma membrane receptor TREM-2 (Bouchon et al., 2001). TREM-2 signals through the ITAM-bearing adaptor DAP-12 to moderately upregulate CD40, CD86 and MHC II, strongly upregulates CCR7, and fails to induce production of inflammatory cytokines or NF-κB and p38 MAP kinase activation (Bouchon et al., 2001). Although the function of TREM-2 or the identity of its ligand are unknown, DAP12^{-/-} mice exhibited accumulation of DCs in muco-cutaneous epithelia as if the emigration from tissues was inhibited (Tomasello et al., 2000). Another intriguing pathway is induced by TSLP (thymic stromal lymphopoietin) (Watanabe et al., 2005). Although first described as a product of inflamed epithelia, TSLP is also produced by Hassall's corpuscles in the thymus, a site of Treg formation. Interestingly, TSLP induces phenotypic maturation but not the release of inflammatory cytokines such as IL12; the DCs so produced can generate CD4⁺CD25⁺ Tregs in vitro.

Physiological role of E-cadherin-induced DC maturation. Although the observation that E-cadherin/β-catenin signaling is likely to play a dramatic regulatory role in DCs is noteworthy, it will next be important to determine the in vivo significance of this pathway. In principle, this can be accomplished using targeted deletions of essential components of the pathway.

The failure of DCs matured in vitro by loss of E-cadherin adhesion to produce inflammatory cytokines suggests that these cells are involved in peripheral tolerance. In support of this possibility are both our own in vivo experiments and recent observations concerning MyD88^{-/-} DCs. Despite exhibiting a typical mature phenotype following LPS treatment, MyD88^{-/-} DCs fail to produce inflammatory cytokines and also fail to activate naïve CD4 T cells in vivo, due to the suppressive effect of Treg's (Pasare and Medzhitov, 2004). Thus, like MyD88-deficient DCs, E-cadherin activation of normal DCs turns on those features of the maturation pathway required for efficient antigen processing and presentation, yet there is a failure of cytokine production, leading to the production of IL10-secreting T cells and possibly tolerance (Menges et al., 2002; O'Garra and Vieira, 2004).

What is known about “tolerogenic” DCs in vivo? It has been elegantly demonstrated that targeting antigens to DCs using antibody to DEC-205 in the absence of overt inflammatory or immunostimulatory mediators led to tolerance, while further maturation by CD40 ligand (CD40L) resulted in immunity (Bonifaz et al., 2002; Hawiger et al., 2001). The steady state DCs present antigens efficiently to drive T cell proliferation, which were then deleted (Hawiger et al., 2001). These “tolerogenic” DCs were phenotypically mature, with their expression of CD40 and CD86 being upregulated only slightly upon further stimulation by CD40L, similar to our observations. More strikingly, while immunization with E-cadherin-matured DCs generated IL-10-producing Tregs consistent with the induction of tolerance, treating these towered DCs with LPS led to strong immunity. Since we could detect the expression of E-cadherin in primary DCs from lymph nodes and peripheral tissues (AJ and IM, unpublished), it is possible that at least some of the DCs in lymphoid organs at steady state had been “matured” by alterations in adhesion.

The maturation program induced by alterations in E-cadherin adhesion may be a component of other maturation pathways. For example, maturation of DCs by pathogens (eg E. coli) does seem to stimulate many of the same transcripts as does CD, while LPS alone (which stimulates only a single TLR, unlike E. coli which stimulates several) fails to produce active β-catenin (FIGS. 3B and 3D). On the other hand, maturation by the E-cadherin pathway appeared to modulate LPS-induced maturation. When LPS was used to stimulate DCs already matured by CD, the induction of Th1 cytokine IL-12p40 was enhanced, while IL-10 release was completely blocked. This latter result is of particular interest in that it suggests the possibility that lymph node DCs, likely already stimulated by E-cadherin activation, can be reactivated to induce enhanced immunity (Th1) and reduce the production of Th2 or regulatory T cell responses (O'Garra and Vieira, 2004). Thus, the E-cadherin-induced DC maturation program appears to be positioned to maintain peripheral tolerance under steady state and to enhance immunity upon pathogen challenge. In light of the recent finding that transcription factor NFAT serves as a common regulator in both the effector T cells and Tr cells (Wu et al., 2006), it is tempting to speculate that alteration of E-cadherin-mediated adhesion activates a similar mediator to determine whether DCs are immunogenic or tolerogenic. In any event, these data demonstrate that the E-cadherin system—despite not being specific to the immune system—is likely to have an important effect on the ability of DCs to control one of the most finely tuned and complex aspects of the immune response.

### Experimental Procedures

Reagents and Antibodies Rat anti-E-cadherin, mouse anti-β-catenin, mouse anti-p120 catenin were purchased from BD Transduction Laboratories (Lexington, Calif.). Both mouse anti-E-cadherin (HECD-1 and SHE78-7) and rat anti-E-cadherin (ECCD-1 and 2) monoclonal antibodies were obtained from Zymed (San Francisco, Calif.). Rat monoclonal anti-E-cadherin and rabbit polyclonal β-catenin were obtained from Sigma-Aldrich. Secondary antibodies for immunofluorescence and FluoroReporter^{R} lacZ flow cytometry kit were from Molecular Probes (Eugene, Oreg.). Anti-active β-catenin antibody was purchased from Upstate Biotechnology (Lake Placid, N.Y.). FACS antibodies: CD86, CD11c, human CD4, mouse CD4, CD8, TCR Vα2, TCR Vβ5, CD25 and MHCII I-A^{b} were from Pharmingen (San Diego, Calif.); CD25, IL2, IFN-γ, FOXP3, and IL10 were from eBioscience (San Diego, Calif.). Antibodies against phospho-specific p38 and IκBa were from Cell Signaling Technology Inc (Beverly, Mass.). SB 216763 was purchased from Tocris Cookson Inc. (Ellisville, Mo.). A PE-Rat anti-mouse CCR7 antibody was purchased from Biolegend (San Diego, Calif.). Brefeldin A (BFA) was purchased from Epicentre. Murine β-catenin-GFP or phosphorylation-mutant β-catenin-GFP was kindly provided by Dr. James Nelson (Stanford University).
Mice C57BL/6 mice were purchased from Charles Rivers Laboratories and CD45.1 C57BL/6, C57BL/10ScCr (TLR4^{-/-}), OT I and OT II TCR transgenic mice were purchased from Jackson Laboratories. The transgenic TOPGAL reporter mice were kindly provided by Dr. E. Fuchs (DasGupta and Fuchs, 1999) (Rockefeller University).
Flow Cytometry Assays Cells were stained for 30 min on ice with primary antibody and if necessary secondary antibody, washed, and then evaluated on a FACSCalibur™ (Becton Dickinson). A fluoReporterR LacZ flow cytometry kit was used to measure β-galatosidase activity for BMDCs from TOPgal transgenic mice following the manufacturer's recommendations. For intracellular staining, splenocytes (1×10⁶ cell/well) were incubated with BFA (5 μg/ml) for the last 6 hours of their in vitro restimulation and surface staining and intracellular staining were performed with BD Cytofix/Cytoperm™ plus kit with manufacturer's protocol.
Cell Culture and purification of DC, OT-I and OT-II T cells Mouse DM-derived CD11c⁺ DCs were isolated using anti-CD11c-conjugated beads and columns (Miltenyi Biotech) according to the manufacturer's protocol. The resulted CD11c⁺ DCs were then resuspended in the same culture media at 5-10×10⁵ cells/ml; these cells were in single cell suspension and thus referred as cluster-disrupted cells. CD4 or CD8 T cells were isolated with CD4 and CD8 T cells isolation kits (Miltenyi Biotech) from lymph nodes according to manufacture's protocol. For proliferation assay, cells were labeled with CFSE (Molecular Probes) at 5 μM at 37° C. for 10 min and washed extensively before injection or plating.
Cell fractionation, Immunoprecipitation and Western blotting Cells were homogenized in hypotonic buffer (10 mM HEPES-KOH pH 7.4, 1.5 mM MgCl2, 10 mM KCl, 0.5 mM DTT) with proteinase and phosphatase inhibitors by passing through 30 gauge needles for 10 times. Postnuclear fractions were centrifuged at 45,000 rpm for 45 min to separate membrane and cytosol fractions. Total cell lysates were obtained with 1% NP-40 lysis buffer (1% Nonidet P-40, 150 mM NaCl, 20 mM Tris, and ImM EDTA (pH 7.5)) supplemented with proteinase and phosphatase inhibitors. For immunoprecipitation, cells were lysed in 1% digitonin and supernatants were then incubated with antibodies and protein G-sepharose at 4° C. with constant rotation for 2-4 hr, captured immune complexes were subjected to SDS-PAGE and Western blotting analysis.
Antigen presentation assay For peptide antigen, CD11c⁺ DCs after different treatments were incubated with OVA-peptide (323-339) at 37° C. for 2-3 hr. The cells were then extensively washed with plain RPMI, fixed with 1% PFA for 10 min and extensively washed before added to 1×10⁵ CD4 T cells freshly purified from OT II lymph nodes. For protein antigen, cells were pulsed with OVA protein (grade VI; Sigma-Aldrich or Worthington) for 2 hr at 37° C. before different treatment. CD11c⁺ DCs were then fixed with 1% PFA before addition to either CD4 or CD8 T cells from OT-11 and OT-I mouse lymph nodes, respectively. Supernatants were taken out after 24 hr incubation at 37° C. and frozen at −70° C. overnight before ELISA assay for IL-2.
Retrovirus generation and transfection of DCs pEGFP-□-catenin and EGFP were cloned into pLZRS and then transfected into □x-ecotropic cells using Fugene 6 (Roche). Retrovirus was generated in □x-ecotropic cells and subsequently used to transfect DCs (Chow et al., 2002). TOP-EGFP and FOP-EGFP, constructed from pTOP/FOPFlash (with minimal c-fos promoter from Dr. H Clevers) were kindly provided by Dr. A. Sartorelli (Yale University). Top/Fop-EGFP were then cloned into the retroviral vector LTRH1 provided by Dr. R. Medzhitov (Yale University) (Barton and Medzhitov, 2002).
Immunofluorescence microscopy Cells were fixed in 4% PFA, permeabilized in RPMI with 10% goat serum and 0.25% saponin followed by 30 min each with primary antibody and secondary antibody with appropriate Alexa® Fluors; (Molecular Probes, Inc.) before mounted in Prolong Gold solution. Confocal microscopy was performed using a laser scanning microscope (LSM 510; Carl Zeiss MicroImaging, Inc.), 40× water immersion lens (n=1.5), at 25° C. Images were processed using Adobe Photoshop® (Adobe Systems, Inc.) version 7.0 and Volocity (Improvision) version 2.6.3 software.
Microarrays and data analysis Total RNA from human CD34⁺ DCs was isolated using RNeasy kits (Qiagen), followed by cDNA synthesis (5 □g RNA per sample) using the SuperScript system (Invitrogen). Samples were then cleaned, prepared and hybridized to Affymetrix (Santa Clara, Calif.) Human Genome U95Av2 arrays (representing approximately 8500 genes) according to manufacturer's protocol. Raw data correction and normalization was performed using Affymetrix Microarray Suite 5.0 for background and PM/MM corrections. The probe set based summary data were then log transformed and normalized for probe set intensity-dependent biases. Loess normalization of M vs. A relationship for all chip-pairs was performed. We considered a gene to be regulated by a treatment only if its expression intensity was increased or reduced by at least 3 fold compared to the intensity measured at time 0. For clustering and heatmap generation, log ratios of expression intensities were standardized within each gene, thereby transforming the distribution of log ratios into one with mean at 0 and standard derivation of 1. Heatmap-associated color scale bars visualize the scaling relationship between color intensities and corresponding standardized log ratio values: up-regulated genes are shown in red while down-regulated genes are shown in blue. Clustering of experimental samples based on transcriptional profiles was carried out using an agglomerative hierarchical clustering algorithm. Log ratio values of all regulated genes were used to construct feature vectors for each sample. A dissimilarity measurement between samples was computed as a Euclidean distance between feature vectors. Cluster dissimilarities were computed using group average method. Unless otherwise specified, all data analysis procedures were implemented using S-Plus software (insightful Corp.).

Cytokine and chemokine multiplex analysis The levels of cytokines and chemokines were measured with Luminex suspension array technology. Supernatants were collected and frozen at −80° C. For human CD34⁺-derived DCs, cell culture supernatants were then analyzed using the Beadlyte cytokine assay kit (Upstate) with manufacturer's protocol. For mouse cytokines, supernatants were analyzed using the Bio-Plex cytokine assay kit and supporting reagents (BioRad) following manufacturer's procedures.

Real-time RT-PCR Total RNA was isolated from differently treated human or murine cells with the RNAeasy kit from Invitrogen according to manufacturer's recommendation. Quantitative real-time RT-PCR was carried out with the DNA Engine Opticon® 2 real time detection system (My Research Inc.) and SYBR Green system (Strategene), and data were normalized by the level of □-actin expression in each individual sample.
Adoptive transfer with DCs and T cells and in vitro restimulation For in vivo proliferation assay, 0.5-1.5×10^{□} labeled or unlabeled purified T cells were injected intravenously into the lateral tail vein of mice, 24 hr later 1×10⁶ DCs pulsed with proper antigens were injected intravenously into the same mice. Adaptive transferred T cells were analyzed for their proliferation 3, 5 or 7 after the last injection. For in vivo CD4 T cell priming, BMDCs were prepared as described and pulsed with OVA peptide 323-339 (10 μg/ml) after maturation treatment for 2 hr at 37° C. and washing extensively afterwards, 1-2.5×10⁶ matured DCs were then injected intravenously at day 0, 2 and 4 and spleen cells were restimulated at day 7 with 10 μg/ml OVA peptide 323-339. Cell supernatants were taken after 72 hr and cytokines were measured as described. For intracellular staining, BFA (5 μg/ml) was added for the last 6 hr of the in vitro restimulation and the cells were fixed and permeabilized using BD Cytofix/Cytoperm™ plus kit.

### REFERENCES

Banchereau, J., and Steinman, R. M. (1998). Dendritic cells and the control of immunity. Nature 392, 245-252.
Barton, G. M., and Medzhitov, R. (2002). Retroviral delivery of small interfering RNA into primary cells. Proc Natl Acad Sci USA 99, 14943-14945.
Barton, G. M., and Medzhitov, R. (2003). Toll-like receptor signaling pathways. Science 300, 1524-1525.
Bonifaz, L., Bonnyay, D., Mahnke, K, Rivera, M., Nussenzweig, M. C., and Steinman, R. M. (2002). J Exp Med 196, 1627-1638.
Bouchon, A., Hernandez-Munain, C., Cella, M., and Colonna, M. (2001). A DAP12-mediated pathway regulates expression of CC chemokine receptor 7 and maturation of human dendritic cells. J Exp Med 194, 1111-1122.
Chow, A., Toomre, D., Garrett, W., and Mellman, I. (2002). Dendritic cell maturation triggers retrograde MHC class II transport from lysosomes to the plasma membrane. Nature 418, 988-994.
Coghlan, M. P., Culbert, A. A., Cross, D. A., Corcoran, S. L., Yates, J. W., Pearce, N. J., Rausch, O. L., Murphy, G. J., Carter, P. S., Roxbee Cox, L., et al. (2000). Selective small molecule inhibitors of glycogen synthase kinase-3 modulate glycogen metabolism and gene transcription. Chem Biol 7, 793-803.
Crabtree, G. R, and Olson, E. N. (2002). NFAT signaling: choreographing the social lives of cells. Cell 109Suppl, S67-79.
DasGupta, R., and Fuchs, E. (1999). Multiple roles for activated LEF/TCF transcription complexes during hair follicle development and differentiation. Development 126, 4557-4568.
Dejana, E. (2004). Endothelial cell-cell junctions: happy together. Nat Rev Mol Cell Biol 5, 261-270.
Delamarre, L., Holcombe, H., and Mellman, I. (2003). Presentation of exogenous antigens on major histocompatibility complex (MHC) class I and MHC class II molecules is differentially regulated during dendritic cell maturation. J Exp Med 198, 111-122.
Doble, D. W., and Woodgett, J. R. (2003). GSK-3: tricks of the trade for a multi-tasking kinase. J Cell Sci 116, 1175-1186.
Geissmann, F., Dieu-Nosjean, M. C., Dezutter, C., Valladeau, J., Kayal, S., Leborgne, M., Brousse, N., Saeland, S., and Davoust, J. (2002). Accumulation of immature Langerhans cells in human lymph nodes draining chronically inflamed skin. J Exp Med 196, 417-430.
Hawiger, D., Inaba, K., Dorsett, Y., Guo, I., Mahnke, K., Rivera, M., Ravetch, J. V., Steinman, R. M., and Nussenzweig, M. C. (2001). Dendritic cells induce peripheral T cell unresponsiveness under steady state conditions in vivo. J Exp Med 194, 769-779.
Jakob, T., Brown, M. J., and Udey, M. C. (1999). Characterization of E-cadherin-containing junctions involving skin-derived dendritic cells. J Invest Dermatol 112, 102-108.
Jakob, T., Ring, J., and Udey, M. C. (2001). Multistep navigation of Langerhans/dendritic cells in and out of the skin. J Allergy Clin Immunol 108, 688-696.
Jakob, T., and Udey, M. C. (1998). Regulation of E-cadherin-mediated adhesion in Langerhans cell-like dendritic cells by inflammatory mediators that mobilize Langerhans cells in vivo. J Immunol 160, 4067-4073.
Kaisho, T., Takeuchi, O., Kawai, T., Hoshino, K, and Akira, S. (2001). Endotoxin-induced maturation of MyD88-deficient dendritic cells. J Immunol 166, 5688-5694.
Korinek V., Barker, N., Morin, P. J., van Wichen, D., de Weger, R, Kinzler, K. W., Vogelstein, B., and Clevers, H. (1997). Constitutive transcriptional activation by a beta-catenin-Tcf complex in APC−/− colon carcinoma. Science 275, 1784-1787.
Lanzavecchia, A., and Sallusto, F. (2001). Antigen decoding by T lymphocytes: from synapses to fate determination. Nat Immunol 2, 487-492.
Lutz, M. B., and Schuler, G. (2002). Immature, semi-mature and fully mature dendritic cells: which signals induce tolerance or immunity? Trends Immunol 23, 445-449.
Lyons, J. P., Mueller, U. W., Ji, H., Everett, C., Fang, X., Hsieh, J. C., Barth, A. M., and McCrea, P. D. (2004). Wnt-1 activates the canonical beta-catenin-mediated Wnt pathway and binds Frizzled-6 CRD: functional implications of Wnt/beta-catenin activity in kidney epithelial cells. Exp Cell Res 298, 369-387.
Martin, M., Rehani, K., Jope, R. S., and Michalek, S. M. (2005). Toll-like receptor-mediated cytokine production is differentially regulated by glycogen synthase kinase 3. Nat Immunol 6, 777-784.
Mellman, I., and Steinman, R. M. (2001). Dendritic cells: specialized and regulated antigen processing machines. Cell 106, 255-258.
Menges, M., Rossner, S., Voigtlander, C., Schindler, H., Kukutsch, N. A., Bogdan, C., Erb, K., Schuler, G., and Lutz, M. B. (2002). Repetitive injections of dendritic cells matured with tumor necrosis factor alpha induce antigen-specific protection of mice from autoimmunity. J Exp Med 195, 15-21.
Merad, M., Manz, M. G., Karsunky, H., Wagers, A., Peters, W., Charo, I., Weissman, I. L., Cyster, J. G., and Engleman, E. G. (2002). Langerhans cells renew in the skin throughout life under steady-state conditions. Nat Immunol 3, 1135-1141.
Nelson, W. J., and Nusse, R. (2004). Convergence of Wnt, beta-catenin, and cadherin pathways. Science 303, 1483-1487.
Norvell, S. M., Alvarez, M., Bidwell, 3. P., and Pavalko, F. M. (2004). Fluid shear stress induces beta-catenin signaling in osteoblasts. Calcif Tissue Int 75, 396-404.
O'Garra, A., and Vieira, P. (2004). Regulatory T cells and mechanisms of immune system control. Nat Med 10, 801-805.
Pasare, C., and Medzhitov, R. (2004). Toll-dependent control mechanisms of CD4 T cell activation. Immunity 21, 733-741.
Pierre, P., Turley, S. J., Gatti, E., Hull, M., Meltzer, J., Mirza, A., Inaba, K., Steinman, R. IL, and Mellman, 1. (1997). Developmental regulation of MHC class II transport in mouse dendritic cells. Nature 388, 787-792.
Randolph, G. J., Sanchez-Schmitz, G., and Angeli, V. (2005). Factors and signals that govern the migration of dendritic cells via lymphatics: recent advances. Springer Semin Immunopathol 26, 273-287.
Rescigno, M., Urbano, M., Valzasina, B., Francolini, M., Rotta, G., Bonasio, R., Granucci, F., Kraehenbuhl, J. P., and Ricciardi-Castagoli, P. (2001). Dendritic cells express tight junction proteins and penetrate gut epithelial monolayers to sample bacteria Nat Immunol 2, 361-367.
Riedl, E., Stockl, J., Majdic, O., Scheinecker, C., Knapp, W., and Strobl, H. (2000a). Ligation of E-cadherin on in vitro-generated immature Langerhans-type dendritic cells inhibits their maturation. Blood 96, 4276-4284.
Riedl, E., Stockl, J., Majdic, O., Scheinecker, C., Rappersberger, K., Knapp, W., and Strobl, H. (2000b). Functional involvement of E-cadherin in TGF-beta 1-induced cell cluster formation of in vitro developing human Langerhans-type dendritic cells. J Immunol 165, 1381-1386.
Romani, N., Holzmann, S., Tripp, C. H., Koch, F., and Stoitzner, P. (2003). Langerhans cells—dendritic cells of the epidermis. Apmis 111, 725-740.
Simons, M., Gloy, J., Ganner, A., Bullerkotte, A., Bashkurov, M., Kronig, C., Schermer, B., Benzing, T., Cabello, O. A., Jenny, A., et al. (2005). Inversin, the gene product mutated in nephronophthisis type II, functions as a molecular switch between Wnt signaling pathways. Nat Genet 37, 537-543.
Sporri, R., and Reis e Sousa, C. (2005). Inflammatory mediators are insufficient for full dendritic cell activation and promote expansion of CD4+ T cell populations lacking helper function. Nat Immunol 6, 163-170.
Staal, F. J., and Clevers, H. C. (2005). WNT signalling and haematopoiesis: a WNT-WNT situation. Nat Rev Immunol 5, 21-30.
Steinman, R. M., Hawiger, D., and Nussenzweig, M. C. (2003). Tolerogenic dendritic cells. Annu Rev Immunol 21, 685-711.
Steinman, R. M., Turley, S., Mellman, I., and Inaba, K. (2000). The induction of tolerance by dendritic cells that have captured apoptotic cells. J Exp Med 191, 411-416.
Takeda, K., and Akira, S. (2004). TLR signaling pathways. Semin Immunol 16, 3-9.
Tang, A., Amagai, M., Granger, L. G., Stanley, J. R., and Udey, M. C. (1993). Adhesion of epidermal Langerhans cells to keratinocytes mediated by E-cadherin. Nature 361, 82-85.
Tomasello, E., Desmoulins, P. O., Chemin, K., Guia, S., Cremer, H., Ortaldo, J., Love, P., Kaiserlian, D., and Vivier, E. (2000). Combined natural killer cell and dendritic cell functional deficiency in KARAP/DAP12 loss-of-function mutant mice. Immunity 13, 355-364.
Trombetta, E. S., and Mellman, I. (2005). Cell biology of antigen processing in vitro and in vivo. Annu Rev Immunol 23, 975-1028.
van Noort, M., Meeldijk, J., van der Zee, R., Destree, O., and Clevers, H. (2002). Wnt signaling controls the phosphorylation status of beta-catenin. J Biol Chem 277, 17901-17905.
Vasioukhin, V., and Fuchs, E. (2001). Actin dynamics and cell-cell adhesion in epithelia. Curr Opin Cell Biol 13, 76-84.
Verbovetski, I., Bychkov, H., Trahtemberg, U., Shapira, I., Hareuveni, M., Ben-Tal, O., Kutikov, I., Gill, O., and Mevorach, D. (2002). Opsonization of apoptotic cells by autologous iC3b facilitates clearance by immature dendritic cells, down-regulates DR and CD86, and up-regulates CC chemokine receptor 7. J Exp Med 196, 1553-1561.
Watanabe, N., Wang, Y. H., Lee, H K, Ito, T., Wang, Y. H., Cao, W., and Liu, Y. J. (2005). Hassall's corpuscles instruct dendritic cells to induce CD4+CD25+ regulatory T cells in human thymus. Nature 436, 1181-1185.
Wu, Y., Borde, M., Heissmeyer, V., Feuerer, M., Lapan, A. D., Stroud, J. C., Bates, D. L., Guo, L., Han, A., Ziegler, S. F., et al. (2006). FOXP3 controls regulatory T cell function through cooperation with NFAT. Cell 126, 375-387.

1. A method of inducing immune tolerance in a patient or subject in need thereof comprising administering to said patient an effective amount of a GSK3 inhibitor.

2. The method according to claim 1 wherein said inhibitor is a GSK-3α, GSK-3β or GSK-3β2 inhibitor.

3. The method according to claim 2 wherein said inhibitor is a GSK3β inhibitor.

4. The method according to claim 1 wherein said GSK3 inhibitor is selected from the group consisting of pyrroloazepines, flavones, benzazepinones, bis-indoles, pyrrolopyrazines, thiadiazolidinones, pyridyloxadiazole, pyrazolopyridines, pyrazolopyridazine, aminopyrimidine, aminopyridine, pyrazoloquinoxalines, oxindoles (Indolinone), thiazoles, bisindolylmaleimides, azaindolylmaleimide, arylindolemaleimides, anilinomaleimides, anilinoarylmaleimides, phenylaminopyrimidines, triazoles, pyrrolopyrimidines, pyrazolopyrimidines, and chloromethylthienylketones.

5. The method according to claim 4 wherein said pyrolloazepine is hymenialdisine; said flavone is flavopiridol, said benzazepinone is kenpaullone, alsterpaullone or azakenpaullone; said bis-indole is indirubin-3′-Oxime, 6-Bromoindirubin-3′-oxime (BIO) or 6-Bromoindirubin-3′-acetoxime; said pyrrolopyrazine is Aloisine A or Aloisine B; said thiadiazolidinones is TDZDB; said pyridyloxadiazole is compound 12 of FIG. 1; said pyrazolopyridine is pyrazolopyridine 18 or pyrazolopyridine 34 of FIG. 1; said pyrazolopyridazine is pyrazolopyridine 9 of FIG. 1; said aminopyrimidine is CHIR98014 or CHIR99021 (CT99021); said aminopyridine is CT20026; said pyrazoloquinoxaline is compound 1 of FIG. 1; said oxindole is SU9516; said thiazoles is ARA014418; said bisindolylmaleimide is staurosporine, compound 5a of FIG. 1; said bisindolylmaleimide is GF109203x or Ro318220IX); said azaindolylmaleimide is compound 29 or compound 46 of FIG. 1; said arylindolemaleimide is SB216763; said anilinomaleimide is SB415286; said anilinoarylmaleimide is compound 15, said phenylaminopyrimidine is CGP60474; said triazoles is compound 8b of FIG. 1; said pyrrolopyrimidines is TWS119; said pyrazolopyrimidine is compound 1A of FIG. 1; and said chloromethylthienylketone is compound 17 of FIG. 1.

6. The method according to claim 1 wherein said GSK3 inhibitor is SB216763 or SB415286.

7. The method according to claim 1 wherein said patient has an autoimmune disease or an immune inflammatory disease.

8. The method according to claim 7 wherein said autoimmune disease or said immune inflammatory disease is systemic lupus erythematosis (SLE), diabetes mellitus (type I), asthma, arthritis, pernicious anemia, or multiple sclerosis.

9. The method according to claim 7 wherein said autoimmune disease or said immune inflammatory disease is an autoimmune blood disease; an autoimmune disease of the musculature; an autoimmune disease of the ear; an autoimmune eye disease, an autoimmune disease of the kidney; an autoimmune skin disease; a cardiovascular autoimmune disease; an endocrine autoimmune disease; an autoimmune gastroenteric disease; an autoimmune nervous disease; and a systemic autoimmune disease.

10. The method according to claim 8 wherein said autoimmune disease is pernicious anemia, autoimmune hemolytic anemia, aplastic anemia, idiopathic thrombocytopenic purpura, ankylosing spondylitis, polymyositis, dermatomyositis, autoimmune hearing loss, Meniere's syndrome, Mooren's disease, Reiter's syndrome, Vogt-Koyanagi-Harada disease, glomerulonephritis, IgA nephropathy; diabetes mellitus (type I), pemphigus, pemphigus vulgaris, pemphigus foliaceus, pemphigus erythematosus, bullous pemphigoid, vitiligo, epidermolysis bullosa acquisita, alopecia areata; autoimmune myocarditis, vasculitis, Churg-Strauss syndrome, giant cells arteritis, Kawasaki's disease, polyarteritis nodosa, Takayasu's arteritis and Wegener's granulomatosis, Addison's disease, autoimmune hypoparathyroidism, autoimmune hypophysitis, autoimmune oophoritis, autoimmune orchitis, Grave's Disease, Hashimoto's thyroiditis, polyglandular autoimmune syndrome type 1 (PAS-1) polyglandular autoimmune syndrome type 2 (PAS-2), and polyglandular autoimmune syndrome type 3 (PAS-3), including autoimmune hepatitis, primary biliary cirrhosis, inflammatory bowel disease, celiac disease, Crohn's disease, including multiple sclerosis, myasthenia gravis, Guillan-Barre syndrome and chronic inflammatory demyelinating neuropathy, including systemic lupus erythematosus, antiphospholid syndrome, autoimmune lymphoproliferative disease, autoimmune polyendocrinopathy, Bechet's disease, Goodpasture's disease, rheumatoid arthritis, osteoarthritis, septic arthritis, sarcoidosis, scleroderma and Sjogren's syndrome.

11. The method according to claim 9 wherein said disease is an autoimmune blood disease.

12. The method according to claim 9 wherein said disease is an autoimmune disease of the musculature.

13. The method according to claim 9 wherein said disease is an autoimmune disease of the ear.

14. The method according to claim 9 wherein said disease is an autoimmune eye disease.

15. The method according to claim 9 wherein said disease is an autoimmune disease of the kidney.

16. The method according to claim 9 wherein said disease is an autoimmune skin disease.

17. The method according to claim 9 wherein said disease is a cardiovascular autoimmune disease.

18. The method according to claim 9 wherein said disease is an endocrine autoimmune disease.

19. The method according to claim 9 wherein said disease is an autoimmune gastroenteric disease.

20. The method according to claim 9 wherein said disease is an autoimmune nervous disease.

21. The method according to claim 9 wherein said disease is a systemic autoimmune disease.

22. The method of claim 9 wherein said disease is systemic lupus erythematosus.

23. The method according to claim 2 wherein said autoimmune disease is diabetes mellitus type I.

24. The method according to claim 9 wherein said disease is arthritis.

25. The method according to claim 9 wherein said disease is multiple sclerosis.

26. A method of treating an autoimmune or immune inflammatory disease in a patient or subject in need of therapy comprising administering to said patient or subject an effective amount of a GSK3 inhibitor.

27. The method according to claim 26 wherein said inhibitor is a GSK-3α, GSK-3β or GSK-3β2 inhibitor.

28. The method according to claim 27 wherein said inhibitor is a GSK3β inhibitor.

29. The method according to claim 1 wherein said GSK3 inhibitor is selected from the group consisting of pyrroloazepines, flavones, benzazepinones, bis-indoles, pyrrolopyrazines, thiadiazolidinones, pyridyloxadiazole, pyrazolopyridines, pyrazolopyridazine, aminopyrimidine, aminopyridine, pyrazoloquinoxalines, oxindoles (Indolinone), thiazoles, bisindolylmaleimides, azaindolylmaleimide, arylindolemaleimides, anilinomaleimides, anilinoarylmaleimides, phenylaminopyrimidines, triazoles, pyrrolopyrimidines, pyrazolopyrimidines, and chloromethylthienylketones.

30. The method according to claim 29 wherein said pyrolloazepine is hymenialdisine; said flavone is flavopiridol, said benzazepinone is kenpaullone, alsterpaullone or azakenpaullone; said bis-indole is indirubin-3′-Oxime, 6-Bromoindirubin-3′-oxime (BIO) or 6-Bromoindirubin-3′-acetoxime; said pyrrolopyrazine is Aloisine A or Aloisine B; said thiadiazolidinones is TDZDB; said pyridyloxadiazole is compound 12 of FIG. 1; said pyrazolopyridine is pyrazolopyridine 18 or pyrazolopyridine 34 of FIG. 1; said pyrazolopyridazine is pyrazolopyridine 9 of FIG. 1; said aminopyrimidine is CHIR98014 or CHIR99021 (CT99021); said aminopyridine is CT20026; said pyrazoloquinoxaline is compound 1 of FIG. 1; said oxindole is SU9516; said thiazoles is ARA014418; said bisindolylmaleimide is staurosporine, compound 5a of FIG. 1; said bisindolylmaleimide is GF109203x or Ro3182201X); said azaindolylmaleimide is compound 29 or compound 46 of FIG. 1; said arylindolemaleimide is SB216763; said anilinomaleimide is SB415286; said anilinoarylmaleimide is compound I5, said phenylaminopyrimidine is CGP60474; said triazoles is compound 8b of FIG. 1; said pyrrolopyrimidines is TWS119; said pyrazolopyrimidine is compound 1A of FIG. 1; and said chloromethylthienylketone is compound 17 of FIG. 1.

31. The method according to claim 26 wherein said GSK3 inhibitor is SB216763 or SB415286.

32. The method according to claim 26 wherein said patient has an autoimmune disease.

33. The method according to claim 26 wherein said patient has an immune inflammatory disease.

34. The method according to claim 26 wherein said autoimmune disease or said immune inflammatory disease is systemic lupus erythematosis (SLE), diabetes mellitus (type I), asthma, arthritis, pernicious anemia, or multiple sclerosis.

35. The method according to claim 26 wherein said autoimmune disease or said immune inflammatory disease is an autoimmune blood disease; an autoimmune disease of the musculature; an autoimmune disease of the ear; an autoimmune eye disease, an autoimmune disease of the kidney; an autoimmune skin disease; a cardiovascular autoimmune disease; an endocrine autoimmune disease; an autoimmune gastroenteric disease; an autoimmune nervous disease; and a systemic autoimmune disease.

36. The method according to claim 35 wherein said autoimmune disease is pernicious anemia, autoimmune hemolytic anemia, aplastic anemia, idiopathic thrombocytopenic purpura, ankylosing spondylitis, polymyositis, dermatomyositis, autoimmune hearing loss, Meniere's syndrome, Mooren's disease, Reiter's syndrome, Vogt-Koyanagi-Harada disease, glomerulonephritis, IgA nephropathy; diabetes mellitus (type I), pemphigus, pemphigus vulgaris, pemphigus foliaceus, pemphigus erythematosus, bullous pemphigoid, vitiligo, epidermolysis bullosa acquisita, alopecia areata; autoimmune myocarditis, vasculitis, Churg-Strauss syndrome, giant cells arteritis, Kawasaki's disease, polyarteritis nodosa, Takayasu's arteritis and Wegener's granulomatosis, Addison's disease, autoimmune hypoparathyroidism, autoimmune hypophysitis, autoimmune oophoritis, autoimmune orchitis, Grave's Disease, Hashimoto's thyroiditis, polyglandular autoimmune syndrome type 1 (PAS-1) polyglandular autoimmune syndrome type 2 (PAS-2), and polyglandular autoimmune syndrome type 3 (PAS-3), including autoimmune hepatitis, primary biliary cirrhosis, inflammatory bowel disease, celiac disease, Crohn's disease, including multiple sclerosis, myasthenia gravis, Guillan-Barre syndrome and chronic inflammatory demyelinating neuropathy, including systemic lupus erythematosus, antiphospholid syndrome, autoimmune lymphoproliferative disease, autoimmune polyendocrinopathy, Bechet's disease, Goodpasture's disease, rheumatoid arthritis, osteoarthritis, septic arthritis, sarcoidosis, scleroderma and Sjogren's syndrome.

37. The method according to claim 35 wherein said disease is an autoimmune blood disease.

38. The method according to claim 35 wherein said disease is an autoimmune disease of the musculature.

39. The method according to claim 35 wherein said disease is an autoimmune disease of the ear.

40. The method according to claim 35 wherein said disease is an autoimmune eye disease.

41. The method according to claim 35 wherein said disease is an autoimmune disease of the kidney.

42. The method according to claim 35 wherein said disease is an autoimmune skin disease.

43. The method according to claim 35 wherein said disease is a cardiovascular autoimmune disease.

44. The method according to claim 35 wherein said disease is an endocrine autoimmune disease.

45. The method according to claim 35 wherein said disease is an autoimmune gastroenteric disease.

46. The method according to claim 35 wherein said disease is an autoimmune nervous disease.

47. The method according to claim 35 wherein said disease is a systemic autoimmune disease.

48. The method of claim 34 wherein said disease is systemic lupus erythematosus.

49. The method according to claim 35 wherein said autoimmune disease is diabetes mellitus type I.

50. The method according to claim 35 wherein said disease is arthritis.

51. The method according to claim 35 wherein said disease is multiple sclerosis.

52. A method of activating an E-cadherin/β-catenin pathway in dendritic cells to produce mature dendritic cells which exhibit a T cell response associated with induction or maintenance of T cell tolerance, rather than immunity, in a patient or subject comprising administering to said patient or subject an effective amount of a GSK3 inhibitor to said patient or subject.

53. The method according to claim 52 wherein said is a GSK-3α, GSK-3β or GSK-3β2 inhibitor.

54. The method according to claim 53 wherein said inhibitor is a GSK3β inhibitor.

55. The method according to claim 1 wherein said GSK3 inhibitor is selected from the group consisting of pyrroloazepines, flavones, benzazepinones, bis-indoles, pyrrolopyrazines, thiadiazolidinones, pyridyloxadiazole, pyrazolopyridines, pyrazolopyridazine, aminopyrimidine, aminopyridine, pyrazoloquinoxalines, oxindoles (Indolinone), thiazoles, bisindolylmaleimides, azaindolylmaleimide, arylindolemaleimides, anilinomaleimides, anilinoarylmaleimides, phenylaminopyrimidines, triazoles, pyrrolopyrimidines, pyrazolopyrimidines, and chloromethylthienylketones.

56. The method according to claim 55 wherein said pyrolloazepine is hymenialdisine; said flavone is flavopiridol, said benzazepinone is kenpaullone, alsterpaullone or azakenpaullone; said bis-indole is indirubin-3′-Oxime, 6-Bromoindirubin-3′-oxime (BIO) or 6-Bromoindirubin-3′-acetoxime; said pyrrolopyrazine is Aloisine A or Aloisine B; said thiadiazolidinones is TDZDB; said pyridyloxadiazole is compound 12 of FIG. 1; said pyrazolopyridine is pyrazolopyridine 18 or pyrazolopyridine 34 of FIG. 1; said pyrazolopyridazine is pyrazolopyridine 9 of FIG. 1; said aminopyrimidine is CHIR98014 or CHIR99021 (CT99021); said aminopyridine is CT20026; said pyrazoloquinoxaline is compound 1 of FIG. 1; said oxindole is SU9516; said thiazoles is ARA014418; said bisindolylmaleimide is staurosporine, compound 5a of FIG. 1; said bisindolylmaleimide is GF109203x or Ro3182201X); said azaindolylmaleimide is compound 29 or compound 46 of FIG. 1; said arylindolemaleimide is SB216763; said anilinomaleimide is SB415286; said anilinoarylmaleimide is compound 15, said phenylaminopyrimidine is CGP60474; said triazoles is compound 8b of FIG. 1; said pyrrolopyrimidines is TWS119; said pyrazolopyrimidine is compound 1A of FIG. 1; and said chloromethylthienylketone is compound 17 of FIG. 1.

57. The method according to claim 52 wherein said GSK3 inhibitor is SB216763 or SB415286.

58-72. (canceled)
